(19) United States
(12) Patent Application Publication
(10) Pub. No.: US 2012/0021014 A1
(43) Pub. Date: Jan. 26, 2012

(54) CORROSION CURRENT-GENERATING METAL PARTICULATES AND USE THEREOF

(76) Inventors: Jeannette Chantalat, Pennington, NJ (US); Jeffrey C. Geesin, Doylestown, PA (US); James E. Hauschild, Cranbury, NJ (US); Julia Hwang, Wayland, MA (US); Wei Kong, Bridgewater, NJ (US); Jue-Chen Liu, Belle Mead, NJ (US); William R. Parrish, Hudson, MA (US); Michael D. Southall, Lawrenceville, NJ (US); Brooks Story, Franklin, MA (US); Ying Sun, Belle Mead, NJ (US); Chunlin Yang, Belle Mead, NJ (US)

(21) Appl. No.: 13/186,816

(22) Filed: Jul. 20, 2011

Related U.S. Application Data
(60) Provisional application No. 61/367,145, filed on Jul. 23, 2010.

(51) Int. Cl. A61K 9/14; A61K 33/06; A61K 33/34; A61K 33/32; A61P 29/00; A61P 17/10; A61P 17/06; A61P 31/00; A61P 41/00; A61K 33/30; A61P 17/00

(52) U.S. Cl. 424/400; 424/642; 424/682; 424/643; 424/641; 424/630; 424/639; 424/59; 424/638

(57) ABSTRACT

Metal particulates capable of generating low levels of corrosion current beneficial for pharmaceutical, cosmetic and other medical uses are provided.

### CROSS REFERENCE TO RELATED APPLICATIONS

This application claims benefit of U.S. Provisional Application No. 61/367,145 filed Jul. 23, 2010. The complete disclosure of the aforementioned related U.S. patent application is hereby incorporated herein by reference for all purposes.

### FIELD OF THE INVENTION

The present invention relates to metal particulates, compositions containing them and their uses in tissue treatment. More particularly, the present invention relates a low, controlled and modulated corrosion current generated by such metal particulates.

### BACKGROUND OF THE INVENTION

It is known that metal corrosion is the disintegration of a metal into its constituent ions and oxides due to electrochemical reactions with its surroundings (e.g., oxygen and aqueous electrolyte). The process of metal corrosion usually results in decay of a metal material or structure, and therefore, is generally considered as undesirable and to be prevented. For example, electrochemical oxidation of elemental iron in reaction with oxygen forms iron oxides commonly known as rust.

Metal corrosion occurs when physical non-uniformities develop on the surface of a metal, creating cathodic and anodic regions on the surface. Such physical non-uniformities include pitting, crevices, mechanical stresses, and inter-granular defects. Kruger, Electrochemistry of Corrosion, Electrochemistry Encyclopedia, (http://electrochem.cwru.edu/encycl/art-c02-corrosion.htm, accessed Jul. 23, 2010), and Zhang, Corrosion and Electrochemistry of Zinc (1996 Plenum Press, New York, pages 217-236). For example, it is known that crevice corrosion occurs when a portion of a metal surface is shielded in such a way that the shielded portion has limited access to the surrounding environment. If the surrounding environment contains an electrolyte such as chloride ions and oxygen, the shielded area is rendered more anodic than the unshielded portion, leading to formation of a crevice and generation of electricity known as corrosion current. Similarly in pit corrosion, tiny pits on the surface of a metal create a relatively anodic region in comparison with the remaining smooth surface of the metal, and corrosion of the metal will therefore occur.

Metal corrosion generally causes adverse effects in metal structures, particularly in medical device such as pins, plates, hip joints, and pacemakers. In situ degradation of metal-alloy implants, for example, is undesirable for two reasons: the degradation process may compromise the structural integrity of the implant, and the release of degradation products may elicit an adverse biological reaction to the host. Corrosion of metal implants leads to device failures through broken connections in pacemakers, inflammation in the tissue surrounding the implants, and fracture of weight-bearing prosthetic devices. Degradation may result from electrochemical dissolution phenomena, physical wear, and/or a synergistic combination of the two. Jacobs et al., Current Concepts Review—Corrosion of Metal Orthopaedic Implants, The Journal of Bone and Joint Surgery (American) 80:268-82, 1998. Since the corrosion rate is proportional to the corrosion current, materials with higher I_{corr} values will corrode more rapidly. A high corrosion rate associated with a high corrosion current density (Amp/cm²) in metal implants may be particularly harmful to the surrounding tissues (e.g., causing inflammation at the corrosion site).

Attempts have been made to control and modulate the corrosion current and corrosion rate of medical devices to provide clinical benefits. For example, US 2006/0229711 describes medical devices that are biodegradable over a clinically relevant period of time to provide the physical and structural function of a medical implant, while preventing the harmful effects associated with metal implant corrosion, such as tissue inflammation at the corrosion sites. The biodegradable medical devices generate a corrosion current density (I_{corr}) ranging from 0.0001 A/cm²to 0.1 A/cm².

The corrosion current/rate of metal implants may be controlled by a variety of means individually or in combination. One means comprises applying a protective coating to slow down the corrosion process and to prevent the metal corrosion-induced tissue inflammation and subsequent undesirable tissue changes (e.g., restinosis after vascular stenting) at the implantation site, as described in US 2006/0229711 and US 2007/0270942.

Galvanic electricity generated from galvanic particulates comprising two or more metals for use on biological tissues has been disclosed in US 2007/0060862 and WO 2009/045720.

As an alternative, applicants have now discovered that metal particulates comprising a single, substantially pure, elemental metal can generate low and advantageous levels of corrosion current that may be used to treat a multitude of conditions, for example those stemming from tissue inflammation, microbial infections (e.g., via bacterial biofilm formation on medical implant surfaces), as well as aid beneficial physiological processes such as wound healing and tissue repair.

### SUMMARY OF THE INVENTION

The invention provides a therapeutic composition comprising single-metal particulates comprising a substantially pure, elemental metal having a Standard Electrode Potential of about −0.6V to about −2.5V and a carrier, said particulates having a particle size of about 10 nanometers to about 500 micrometers and being capable of generating a current density of less than 100 microA/cm².

In one embodiment, the invention also provides a method of treating mammalian tissue, which comprises administering to said mammalian tissue a composition comprising single-metal particulates comprising a substantially pure, elemental metal having a Standard Electrode Potential of about −0.6V to about −2.5V and a carrier, said particulates having a particle size of about 10 nanometers to about 500 micrometers and being capable of generating a current density of less than 100 microA/cm².

In another embodiment, the invention further provides a method of treating a skin condition in a mammal, which comprises topically applying to skin having such skin condition a composition comprising single-metal particulates comprising a substantially pure, elemental metal having a Standard Electrode Potential of about −0.6V to about −2.5V and a topical carrier, said particulates having a particle size of about 10 nanometers to about 500 micrometers and being capable of generating a current density of less than 100 microA/cm².

In another embodiment, the invention provides a method of preventing tissue adhesion, which comprises applying to mammalian tissue that has been subjected to trauma or surgery a composition comprising single-metal particulates comprising a substantially pure, elemental metal having a Standard Electrode Potential of about −0.6V to about −2.5V and a carrier, said particulates having a particle size of about 10 nanometers to about 500 micrometers and being capable of generating a current density of less than 100 microA/cm².

In another embodiment, the invention provides a method of reducing arthritis pain, which comprises applying to a joint suffering from arthritis a composition comprising single-metal particulates comprising a substantially pure, elemental metal having a Standard Electrode Potential of about −0.6V to about −2.5V and a carrier, said particulates having a particle size of about 10 nanometers to about 500 micrometers and being capable of generating a current density of less than 100 microA/cm².

In another embodiment, the invention provides a method of reducing inflammation, which comprises applying to mammalian tissue suffering from inflammation a composition comprising single-metal particulates comprising a substantially pure, elemental metal having a Standard Electrode Potential of about −0.6V to about −2.5V and a carrier, said particulates having a particle size of about 10 nanometers to about 500 micrometers and being capable of generating a current density of less than 100 microA/cm².

In another embodiment, the invention provides a method of treating microbial infection, which comprises applying to mammalian tissue suffering from microbial infection a composition comprising single-metal particulates comprising a substantially pure, elemental metal having a Standard Electrode Potential of about −0.6V to about −2.5V and a carrier, said particulates having a particle size of about 10 nanometers to about 500 micrometers and being capable of generating a current density of less than 100 microA/cm².

### DETAILED DESCRIPTION OF THE INVENTION

Unless defined otherwise, all technical and scientific terms used herein have the same meaning as commonly understood by one of ordinary skill in the art to which the invention pertains. Also, all publications, patent applications, patents, and other references mentioned herein are incorporated by reference. Unless otherwise indicated, a percentage refers to a percentage by weight, i.e., % (W/W).

As used herein, “therapeutic” means for the treatment of a disease or condition of mammalian tissue. A therapeutic composition may for example be a cosmetic, personal care, device, pharmaceutical, over-the counter, prescription, or veterinary product.

As used herein, the term “pharmaceutically-acceptable,” “dermatologically-acceptable,” or cosmetically-acceptable” means that the ingredients which the term describes are suitable for use in contact with mammalian tissue (e.g., the skin or mucosa) without undue toxicity, incompatibility, instability, irritation, allergic response, and the like.

As used herein, the term “safe and effective” means sufficient to provide the desired benefit at a desired level, but low enough to avoid serious side effects. The safe amount of the ingredient or composition will vary with the area being treated, the age and tissue of the patient, the duration and nature of the treatment, the specific ingredients or composition employed, the particular carrier utilized, and like factors.

As used herein, the terms “treat” or “treatment” means the treatment (e.g., alleviation or elimination of symptoms and/or cure) and/or prevention or inhibition of a disease or condition.

As used herein, “mammalian tissue” means tissue of a human or other mammal, including internal tissues (muscle, nerve, bone and connective tissues), external tissues such as barrier membranes, or mucosal membranes, such as oral, rectal, or vaginal mucosal membranes. Mammalian tissue includes soft tissues (e.g., the skin, mucosa, epithelium, wound, eye and its surrounding tissues, cartilage and other soft musculoskeletal tissues such as ligaments, tendons, or meniscus), hard tissues (e.g., bone, teeth, nail matrix, or hair follicle), and soft tissue-hard tissue conjunctions (e.g., conductive tissues around periodontal area involved teeth, bones or soft tissue of the joint).

As used herein, the term “barrier membrane” means the thin layer of tissue which covers a surface thereby separating cellular structures or organs. Barrier membrane includes, without limitation, epidermis or epithelial tissue. As used herein, the term “skin” means all external surfaces of a patient, such as the exposed hide or surfaces covered by hair.

The term “patient” refers to a mammal which is being treated. Preferably the patient is a human. However, the compositions and methods of the invention are also suitable for treatment animals.

### Metal Particulates

The present invention utilizes particulates comprising a single, substantially pure, elemental metal that are capable of generating a corrosion current (“particulates” or “metal particulates”). The particulates may be formulated into compositions, such as topical, ingestible, or injectible compositions, or coated onto medical devices, or combined with medical devices, to provide products having a wide variety of pharmaceutical and cosmetic benefits. The invention also relates to methods of utilizing the corrosion current generated from such particulates for treatment of humans and other mammals.

The corrosion current generated by the particulates is caused by a cathodic/anodic reaction that occurs on the surface of the elemental metal. Anode and cathode regions form on the surface of the metal particulates due to a difference in physical characteristics or physical defect between the two regions, such as shape differences, smoothness differences, partial coverage by an essentially non-conductive layer (e.g., oxides, sulfides, phosphates of the metal).

The particulates comprise a substantially pure, elemental metal. As used herein, “elemental” means the valence of the metal is zero. As used herein, “substantially pure” means the metal contains less than 10% by weight, preferably less than 5% by weight, more preferably less than 1% by weight, most preferably less than 0.1% by weight, of other metals or impurities. In one embodiment, the particulates are in the form of a mono-phase alloy. The mono-phase alloy comprises the elemental metal as the primary metal along with a small amount (i.e., less than about 5%, preferably less than about 1%, by weight) of one or more secondary elemental metals. The secondary elemental metal is also elemental, i.e., in the zero valence state.

As used herein, “mono-phase” is defined as a single phase material, i.e., uniform and having no distinct phase boundaries.

As used herein, “mono-phase alloy” is defined as an alloy of two or more metals in the form of a solid solution (i.e., having a uniform distribution of different atoms), i.e., an alloy having a mono-phase.

In one embodiment, the Standard Electrode Potential of the metal ranges from about −0.6V to about −2.5V. Preferably, the metal is selected from the group consisting of magnesium (Standard Electrode Potential of Mg=−2.37V), aluminum (Standard Electrode Potential of Al=−1.66V), and zinc (Standard Electrode Potential of Zn=−0.76V). More preferably, the metal is zinc or magnesium.

In one embodiment, the particulates react with environmental oxygen to form a very thin, partial, or porous coating of metallic oxide layer on the surfaces, which upon contact with an electrolyte, such as a body fluid (e.g., interstitial fluid, wound exudates, blood, sweat, gastrointestinal fluid, etc.), generates corrosion current via crevice corrosion or pitting corrosion as described above.

In one embodiment, the corrosion current density generated by the particulates (i.e., micro-Amp per unit area of tissue surface exposed to the particulates, i.e., microA/cm²) is less than 100 microA/cm², preferably, less than 50 microA/cm², and more preferably, less than 10 microA/cm².

In one embodiment, the particulates comprise substantially pure elemental metals. Such elemental metals include zinc, magnesium, and aluminum.

In another embodiment, the particulates comprise a mono-phase alloy comprising an elemental metal selected from the group of zinc, magnesium and aluminum, and a secondary elemental metal selected from the group consisting of copper, iron, manganese, selenium and mixtures thereof

In another embodiment, the particulates comprise a mono-phase alloy comprising a mixture of two elemental metals both selected from the group of zinc, magnesium and aluminum,

The particulates may be made in accordance with methods of manufacturing known in the art of metal powder processing, such as the methods described in the book, Asm Handbook Volume 7: Powder Metal Technologies and Applications (Asm International Handbook Committee, edited by Peter W. Lee, 1998). For example, the particulates may be produced by atomization processes, including water atomization, oil atomization, and gas atomization. Other atomization methods include centrifugal atomization and ultrasonic/vibrational atomization (pages 35-52 and 72-79), and milling process (pages 53-71).

The particle size of particulates is sufficiently fine to be suspended in a semi-solid form during storage. The average particle size of the particulates is from about 10 nanometers to about 500 micrometers, preferably, from about 100 nanometers to about 100 micrometers. The particle size, as used herein, refers to the maximum dimension in at least one direction.

In one embodiment, the particulates are in flattened and/or elongated shapes. The advantages of flattened and elongated shapes of the particulates include a lower apparent density and, therefore, a better floating/suspending capability in the topical formulation, as well as better coverage over the biological tissue, leading to a wider and/or deeper range of the corrosion current density passing through the biological tissue (e.g., the skin or mucosa membrane). In one embodiment, the longest dimension of the particulates is at least twice of the shortest dimension of such particulates. The particulates may be of any shape, including but not limited to, spherical or non-spherical particles or elongated or flattened shapes (e.g., cylindrical, fibers or flakes).

In another embodiment, the particulates of the present invention may also be coated with other compound materials (i.e., as chemical compounds rather than elemental metals, such as oxides, halides, phosphates, sulfides, etc.) to protect the particulates from degradation during storage (e.g., oxidation degradation from oxygen and moisture), or to modulate the electrochemical reactions and to control the electric current generate when in use. The exemplary coating materials over the material(s) are inorganic or organic polymers, natural or synthetic polymers, biodegradable or bioabsorbable polymers, silica, glass, various metal oxides (e.g., oxide of zinc, aluminum, magnesium, or titanium) and other inorganic salts of low solubility (e. g, zinc phosphate). The weight ratio of the compound coating material to the particulate core is typically less than 1:1, preferably less than 1:5, and most preferably less than 1:10. The coating methods are known in the art of metallic powder processing and metal pigment productions, as described by U.S. Patent publications U.S. Pat. No. 5,964,936; U.S. Pat. No. 5,993,526; U.S. Pat. No. 7,172,812; US 20060042509A1 and US 20070172438.

In one embodiment, the particulates are stored in anhydrous forms, e.g., as a dry powder or immobilized in a fabric with binding agents, or as an essentially anhydrous non-conducting organic solvent composition (e.g., dissolved in polyethylene glycols, propylene glycol, glycerin, liquid silicone, and/or alcohol). In another embodiment, the particulates are embedded into the anhydrous carrier (e.g., inside a polymer) or coated onto a substrate (e.g., as a coating or in the coating layer of a healthcare product such as wound dressing or dental floss). In yet another embodiment, the particulates are encapsulated in compositions of microcapsules, liposomes, micelles, or embedded in the lipophilic phase of oil-in-water (O/W) or water-in-oil (W/O) types of emulsion systems (e.g., W/O lotion, W/O ointment, or O/W creams, where the oil phase can be plant-based oil, mineral-based oil, natural or synthetic oils including silicones of various structures), as well as self-emulsifying compositions, in order to achieve self-life stability, retard the activation of the particulates, or prolong the action of particulates.

In another embodiment, the particulates are provided with a carrier comprising an electrolyte.

### Methods of Use of Particulates

In one embodiment, the corrosion current generated by the metal particulates is used to treat tissues of mammals including humans, by applying to the external surface of the human body (i.e., topical applications onto the skin) or body cavities (e.g., oral, nasal, ear, eye, vaginal and anal, etc.), or internal applications such as gastrointestinal, injection, implantation, open and endoscopic surgical procedures.

The particulates may be contained in a wide variety of cosmetic, therapeutic, or pharmaceutical compositions discussed below. The particulates may be applied directly to a target location of the body in need of such a therapeutic treatment (e.g., either topically or inside the body).

The particulates may be used to treat a variety of conditions, disorders and diseases, such as but not limited to, antimicrobial infection, inflammation, tissue regeneration and tissue repair and healing (e.g., soft tissues such as dermal and sub-dermal tissues, muscles, epithelial, tendon, hard tissue such as bone, tooth, connective tissue , dermal and deep tissue wounds, bone fracture), preventing or reducing body surface or internal scarring, increasing cellular synthesis of extracellular matrix materials (e.g., collagen and elastin), preventing or reducing tissue pigmentation, promoting hair growth (e.g., scalp hair, eye brow and eye lashes).

The composition may be administered to a human or other mammal by any means used in the pharmaceutical or cosmetic arts, including topical administration, oral (including ingestible) administration, parenteral administration (including injection or implantation), nasal administration, intravaginal administration, and the like. In another embodiment, the composition can be administered by injection directly into the target area, such as intra-articularly. Administration may be local or systemic.

Accordingly, the metal particulates can be used in many consumer and medical products for human and animal applications such as in ingestible compositions (such as tablets and solutions), topical compositions (such as creams, lotions, gels, shampoos, cleansers, powders patches, bandages, and masks for application to the skin or mucosal membranes), garments (such as undergarments, underwear, bras, shirts, pants, pantyhose, socks, head caps, facial masks, gloves, and mittens), linens (such as towels, pillow covers or cases and bed sheets), and personal and medical products (such as sanitizing products for household and clinical settings, microcides for plants) and devices (such as toothbrushes, dental flosses, periodontal implants or inserts, orthodontic braces, joint wraps/supports, buccal patches, ocular inserts or implants such as contact lenses, nasal implants or inserts, and contact lens cleaning products, wound dressings, diapers, sanitary napkins, and wipes, tampons, rectal and vaginal suppositories), and coatings or embedded surfaces on medical devices and other surfaces where the anti-inflammatory effects are desired.

Compositions containing the particulates may alternatively be made into a wide variety of products for application on mucosal membranes, including but not limited to vaginal creams, tampons, suppositories, floss, mouthwash, or toothpaste. Other product forms can be formulated by those of ordinary skill in the art.

In one embodiment, the particulates are incorporated into a wound dressing or bandage.

In another embodiment, the particulates are incorporated into a transdermal drug delivery.

In one embodiment, the particulates induce certain desirable biological responses that facilitate the treatment of a barrier membrane condition (e.g., by the corrosion current passing through the barrier membrane and/or by enhancing the delivery of an active agent accompanying the particulates). In one embodiment, the particulates provide multiple mechanisms of action to treat conditions, such as by enhance delivery of active agents by iontophoresis and/or electro-osmosis as well as providing electric stimulation to treat the contacted tissue (e.g., to increase blood circulation or other benefits).

The particulates can be combined with an active agent (such as antimicrobial agents, anti-inflammatory agents, and analgesic agents) to enhance or potentiate the biological or therapeutic effects of that active agent. What is meant by an “active agent” is a compound (e.g., a synthetic compound or a compound isolated from a natural source) that has a cosmetic or therapeutic effect on the barrier membrane or the surrounding tissues (e.g., a material capable of exerting a biological effect on a human body) such as therapeutic drugs or cosmetic agents. Examples of such therapeutic drugs include small molecules, peptides, proteins, nucleic acid materials, and nutrients such as minerals and extracts. The amount of the active agent will depend on the nature of the active agent, the particulates, and/or the intended use of the composition or product.

In one embodiment, a composition containing the particulates further contains a safe and effective amount of an active agent, for example, from about 0.001 percent to about 20 percent, by weight, such as from about 0.01 percent to about 10 percent, by weight, of the composition.

In another embodiment, the particulates can also be combined with other substances to enhance or potentiate the activity of the particulates. Substances that can enhance or potentiate the activity of the particulates include, but are not limited to, organic solvents (such as alcohols, glycols, glycerin, polyethylene glycols and polypropylene glycol), surface active agents (such as nonionic surfactants, zwitterionic surfactants, anionic surfactants, cationic surfactants and polymeric surfactants), and water-soluble polymers. For example, the particulates of the present invention can form conjugates or composites with synthetic or natural polymers including by not limited to proteins, polysaccharides, hyaluronic acid of various molecular weight, hyaluronic acid analogs, polypeptides, and polyethylene glycols.

In one embodiment, a composition comprising the metal particulates contains a chelator or chelating agent. Examples of chelators include, but are not limited to, amino acids such as glycine, lactoferrin, edetate, citrate, pentetate, tromethamine, sorbate, ascorbate, deferoxamine, derivatives thereof, and mixtures thereof. Other examples of chelators useful are disclosed in U.S. Pat. No. 5,487,884 and PCT Publication Nos. 91/16035 and 91/16034.

In one embodiment, the particulates are used to provide intended therapeutic electric stimulation effects by applying the particulates directly to a target location of the body in need such a therapeutic treatment (e.g., either topically or inside the body), including soft tissues, hard tissues, and soft tissue-hard tissue conjunctions.

Therapeutic effects obtained from treatment with the metal particulates include, but are not limited to: antimicrobial effects (e.g., antibacterial, antifungal, antiviral, and anti-parasitic effects); anti-inflammation effects including effects in the superficial or deep tissues (e.g., reduce or elimination of soft tissue edema or redness); elimination or reduction of pain, itch or other sensory discomfort (e.g., headache, sting or tingling numbness); regeneration or healing enhancement of both soft and hard tissues; modulation of stem cell differentiation and tissue development such as modulation of tissue growth (e.g., enhancing growth rate of the nail or regrowth of hair loss due to alopecia) or increase soft tissue volume (e.g., increasing collagen or elastin in the skin or lips); increasing adipocyte metabolism or improving body appearance (e.g., effects on body contour or shape); and increasing circulation of blood or lymphocytes.

As used herein, the terms “inflammatory disorders” and “inflammation” generally mean a reaction of mammalian tissue to irritation, infection, or injury. “Clinical inflammation” can appear as visible redness (erythema), swelling (edema), or as a bruise (contusion). “Subclinical inflammation” refers to the phase of inflammation prior to the manifestation of visible symptoms. Subclinical inflammation is a low level of inflammation characterized by an elevated level of free radicals and pro-inflammatory proteins.

Inflammatory disorders and related conditions include, but are not limited to, arthritis, bronchitis, contact dermatitis, atopic dermatitis, psoriasis, seborrheic dermatitis, eczema, allergic dermatitis, polymorphous light eruptions, inflammatory dermatoses, folliculitis, alopecia, poison ivy, insect bites, acne inflammation, rosacea inflammation, skin or mucosal condition of irritation, edema, itch or pain. Specifically, the inflammatory disorders and related conditions are arthritis, inflammatory dermatoses, contact dermatitis, allergic dermatitis, atopic dermatitis, polymorphous light eruptions, irritation, including erythemas induced by extrinsic factors, acne inflammation, psoriasis, seborrheic dermatitis, eczema, poison ivy, insect bites, folliculitus, alopecia, and secondary conditions and the like. Secondary conditions resulting from inflammation include, but not limited to, xerosis, hyperkeratosis, pruritus, post-inflammatory hyperpigmentation, scarring and the like.

One skilled in the art will recognize that, both in vivo and in vitro trials using suitable, known and generally accepted cell and/or animal models are predictive of the ability of an ingredient, composition, or product to treat or prevent a given condition. One skilled in the art will further recognize that human clinical trials including first-in-human, dose ranging and efficacy trials, in healthy patients and/or those suffering from a given condition or disorder, may be completed according to methods well known in the clinical and medical arts.

### Ingestible Compositions

In one embodiment, the invention provides an ingestible composition containing metal particulates. In one embodiment, the ingestible compositions herein contain, per unit dosage unit, about 1 mg to about 1 g of the metal particulates, such as from about 5 mg to about 500 mg, and may be given at a dosage of from about 0.1 mg/kg/day to about 0.1 g/kg/day, such as from about 0.5 to about 50 mg/kg/day. The dosages, however, may be varied depending upon the requirement of the patient, the severity of the condition being treated, and the metal and active agent being employed. For example, for zinc, the oral dose can be up to 40 mg per day, whereas for magnesium, the oral dose can be up to 400 mg per day. The use of either daily administration or post-periodic dosing may be employed. In one embodiment, these compositions are in unit dosage forms from such as tablets, pills, capsules, powders, granules, solutions or suspensions, and drops.

In one embodiment, the ingestible compositions are provided in the form of tablets, such as those containing 1, 5, 10, 25, 50, 100, 150, 200, 250, 500, and/or 1000 milligrams of the particulates. The composition may be administered on a regimen of 1 to 4 times per day. Advantageously, the composition may be administered in a single daily dose, or the total daily dosage may be administered in divided doses of two, three or four times daily.

Optimal dosages to be administered may be readily determined by those skilled in the art, and will vary with the particular metal particulate used, the mode of administration, the strength of the preparation, and the advancement of the disease/condition being treated. In addition, factors associated with the particular patient being treated, including patient age, weight, diet and time of administration, will result in the need to adjust dosages.

Ingestible compositions containing one or more types of the metal particulates described herein can be prepared by intimately mixing the same with a pharmaceutically-acceptable carrier according to conventional pharmaceutical compounding techniques. The carrier may take a wide variety of forms depending upon the type of formulation. Thus for liquid preparations such as suspensions, elixirs and solutions, suitable carriers and additives include but not limited to water, glycols, alcohols, silicones, waxes, flavoring agents, buffers (such as citrate buffer, phosphate buffer, lactate buffer, gluconate buffer), preservatives, stabilizers, coloring agents and the like; and for solid preparations, such as powders, capsules and tablets, suitable carriers and additives include starches, sugars, diluents, granulating agents, lubricants, binders, disintegrating agents and the like. Solid oral preparations may also be coated with substances such as sugars, soluble polymer film, and insoluble-but-solute permeable polymer film. Oral preparations may also be coated with enteric coatings, which are not soluble in the acidic stomach environment but will dissolve in the intestine as the pH becomes neutral, so as to adjust the site of administration of the agent.

For preparing solid compositions such as tablets, the metal particulate is mixed with a pharmaceutically-acceptable carrier, e.g., conventional tableting ingredients such as corn starch, lactose, sucrose, sorbitol, talc, stearic acid, magnesium stearate, dicalcium phosphate or gums, and other pharmaceutically-acceptable diluents, to form a solid preformulation composition containing a homogeneous mixture. When referring to these preformulation compositions as homogeneous, it is meant that the particulates are dispersed evenly throughout the composition so that the composition may be readily subdivided into equally effective dosage forms such as tablets, pills and capsules. This solid preformulation composition may then subdivided into unit dosage forms of the type described above. The tablets or pills of the composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids with such materials as shellac, cetyl alcohol and cellulose acetate.

In one embodiment, ingestible compositions containing particulates are used for the treatment of gastrointestinal disorders, such as ulcers, diarrhea, and gastrointestinal pain.

In one embodiment, the particulates can be combined with active agents known to treat diarrhea which include, but are not limited to: bismuths (such as Bismuth Subsalicylate), Loperamide, Simethicone, Nitazoxanide, Ciprofloxacin, and Rifaximin, salts and prodrugs (such as esters) thereof

In one embodiment, the particulates can be combined with active agents known to treat gastric ulcers which include, but are not limited to: Lansoprazole, Naproxen, Esomeprazole, Famotidine, Nizatidine, Ranitidine, and Omeprazole, and salts and prodrugs thereof

In one embodiment, the particulates can be combined with active agents known to treat intra-abdominal infections which include, but are not limited to: Moxifloxacin, Ciprofloxacin, Ceftazidime, Gentamicin, Ertapenem; Cefepime, Cefoxitin, Cilastatin, Imipenem; Ceftriaxone, Clavulanate, and Ticarcillin, and salts and prodrugs thereof.

In one embodiment, ingestible compositions containing the particulates are used for treatment of pain (such as throat pain). Oral dosage forms can be in the forms of, but not limited to, lozenges or liquids. Particulates can be combined with active agents known to treat sore throat, which include, but are not limited to: Acetaminophen, Dextromethorphan, Pseudoephedrine, Chlorpheniramine, Pseudoephedrine, Guaifenesin, Doxylamine, Zinc, and Ibuprofen, and salts and prodrugs thereof

In one embodiment, ingestible compositions containing the particulates are used as oral supplements or complements to oral supplements. Oral supplements can be in the forms of, but not limited to, lozenges, tablets, caplets, powders, or liquids. The particulates can be combined with oral supplements of vitamins and minerals, which include, but are not limited to: Dibasic Calcium Phosphate, Magnesium Oxide, Potassium Chloride, Microcrystalline Cellulose, Ascorbic Acid (Vit. C), Ferrous Fumarate, Calcium Carbonate, dl-Alpha Tocopheryl Acetate (Vit. E), Acacia, Ascorbyl Palmitate, Beta Carotene, Biotin, BHT, Calcium Pantothenate, Calcium Stearate, Chromic Chloride, Citric Acid, Crospovidone, Cupric Oxide, Cyanocobalamin (Vit. B 12), Ergocalciferol (Vit. D), Folic Acid, Gelatin, Hypromellose, Lutein, Lycopene, Magnesium Borate, Magnesium Stearate, Manganese Sulfate, Niacinamide, Nickelous Sulfate, Phytonadione (Vit. K), Potassium Iodide, Pyridoxine Hydrochloride (Vit. B), Riboflavin (Vit. B 2), Silicon Dioxide, Sodium Aluminum Silicate, Sodium Ascorbate, Sodium Benzoate, Sodium Borate, Sodium Citrate, Sodium Metavanadate, Sodium Molybdate, Sodium Selenate, Sorbic Acid, Stannous Chloride, Sucrose, Thiamine Mononitrate (Vit. B 1), Titanium Dioxide, Tribasic Calcium Phosphate, Vitamin A Acetate (Vit. A), and Zinc Oxide, and salts and prodrugs thereof. In addition, in one embodiment, the metal particulates can serve as mineral supplements generated in situ, e.g. zinc metal converted to zinc ion in situ.

### Topical Compositions

In one embodiment, the invention provides topical compositions containing the metal particulates that are suitable for administering to mammalian skin, such as human skin. In one embodiment, the topical composition contains (i) the particulates and (ii) a topical carrier. The topical composition may contain a wide variety of active agents depending on the desired use, as described below.

The topical composition may be made into or incorporated in a wide variety of products that include but are not limited to leave-on products (such as lotions, creams, gels, sticks, sprays, and ointments), skin cleansing products (such as liquid washes, solid bars, and wipes), hair products (such as shampoos, conditioners, sprays, and mousses), shaving creams, film-forming products (such as masks), make-up (such as foundations, eye liners, and eye shadows), deodorant and anti-perspirant compositions, and the like. These product types may contain several types of pharmaceutically- or cosmetically-acceptable carriers including, but not limited to solutions, suspensions, emulsions such as microemulsions and nanoemulsions, gels, and solids carrier forms. Other product forms can be formulated by those of ordinary skill in the art.

The topical composition comprises a pharmaceutically-acceptable or cosmetically-acceptable, topical carrier. The topical carrier should not only be compatible with the particulates and any additional active ingredients contained therein, but should not introduce any toxicity and safety issues. The amount of topical carrier varies from about 50% to about 99% by weight of the topical composition of this invention, more preferably from about 75% to about 99% of the composition and most preferably from about 85% to about 95% by weight of the composition.

In one embodiment, the composition is in anhydrous form, e.g., as a cosmetic powder or stick composition, comprising an anhydrous carrier, an essentially anhydrous non-conducting organic solvent composition (e.g., dissolved or suspended in polyethylene glycols, propylene glycol, glycerin, liquid or semisolid silicones, and/or alcohol). In another embodiment, the composition is embedded into an anhydrous carrier (e.g., inside a polymer) or coated onto a substrate (e.g., as a coating or in the coating layer of a healthcare product such as wound dressing or dental floss). In yet another embodiment, composition or metal particulates are encapsulated in microcapsules, liposomes, micelles, or embedded in the lipophilic phase of oil-in-water (O/W) or water-in-oil (W/O) types of emulsion systems (e.g., W/O lotion, W/O ointment, or O/W creams), as well as self-emulsifying compositions, in order to achieve self-life stability or to prolong the action of composition.

Examples of surface active agents which may be used in the topical compositions of this invention include sodium alkyl sulfates, e.g., sodium lauryl sulfate and sodium myristyl sulfate, sodium N-acyl sarcosinates, e.g., sodium N-lauroyl sarcosinate and sodium N-myristoyl sarcosinate, sodium dodecylbenzenesulfonate, sodium hydrogenated coconut fatty acid monoglyceride sulfate, sodium lauryl sulfoacetate and N-acyl glutamates, e.g., N-palmitoyl glutamate, N-methylacyltaurin sodium salt, N-methylacylalanine sodium salt, sodium a-olefin sulfonate and sodium dioctylsulfosuccinate; N-alkylaminoglycerols, e.g., N-lauryldiaminoethylglyecerol and N-myristyldiaminoethylglycerol,N-alkyl-N-carboxymethylammonium betaine and sodium 2-alkyl-1-hydroxyethylimidazoline betaine; polyoxyethylenealkyl ether, polyoxyethylenealkylaryl ether, polyoxyethylenelanolin alcohol, polyoxyethyleneglyceryl monoaliphatic acid ester, polyoxyethylenesorbitol aliphatic acid ester, polyoxyethylene aliphatic acid ester, higher aliphatic acid glycerol ester, sorbitan aliphatic acid ester, Pluronic type surface active agent, and polyoxyethylenesorbitan aliphatic acid esters such as polyoxyethylenesorbitan monooleate and polyoxyethylenesorbitan monolaurate. Emulsifier-type surfactants known to those of skill in the art may be used in the topical compositions of this invention.

In one embodiment, the topical composition is used for the treatment of a skin condition. Examples of skin conditions include, but are not limited to: acne (e.g., blackheads and whiteheads), rosacea, nodule-cystic, and other microbial infections of the skin; visible signs of skin aging (e.g., wrinkles, sagging, sallowness, and age-spots); lax or sagging skin; folliculitis and pseudo-folliculitis barbae; excess sebum (e.g., for sebum reduction or oily/shining skin appearance inhibition or control); excess pigmentation (e.g., reduction of hyperpigmentation such as freckles, melasma, actinic and senile lentigines, age-spots, post-inflammatory hypermelanosis, Becker's naevus, and facial melanosis or enhancing the pigmentation of light skin); excess hair growth (e.g., skin on the leg) or insufficient hair growth (e.g., on the scalp); dermatitis (e.g., atopic, contact, or seborrheic dermatitis), dark circles under the eyes, stretch marks, cellulite, excessive sweating (e.g., hyperhidrosis), and/or psoriasis.

### Topical Anti-Acne/Anti-Rosacea Products

In one embodiment, the topical composition comprises an anti-acne and/or anti- rosacea active agent. Examples of anti-acne and anti-rosacea agents include, but are not limited to: retinoids such as tretinoin, isotretinoin, motretinide, adapalene, tazarotene, azelaic acid, and retinol; salicylic acid; benzoyl peroxide; resorcinol; sulfur; sulfacetamide; urea; antibiotics such as tetracycline, clindamycin, metronidazole, and erythromycin; anti inflammatory agents such as corticosteroids (e.g., hydrocortisone), ibuprofen, naproxen, and ketoprofen; and imidazoles such as ketoconazole and elubiol; and salts and prodrugs thereof.

Other examples of anti-acne active agents include essential oils, alpha-bisabolol, dipotassium glycyrrhizinate, camphor, β-glucan, allantoin, feverfew, flavonoids such as soy isoflavones, saw palmetto, chelating agents such as EDTA, lipase inhibitors such as silver and copper ions, hydrolyzed vegetable proteins, inorganic ions of chloride, iodide, fluoride, and their nonionic derivatives chlorine, iodine, fluorine, and synthetic phospholipids and natural phospholipids such as Arlasilk™ phospholipids CDM, SV, EFA, PLN, and GLA (Uniqema, ICI Group of Companies, Wilton, UK).

### Topical Anti-Aging Products

In one embodiment, the topical composition or product contains an anti-aging active agent. Examples of suitable anti-aging agents include, but are not limited to: inorganic sunscreens such as titanium dioxide and zinc oxide; organic sunscreens such as octyl-methoxy cinnamates; retinoids; dimethylaminoathanol (DMAE), copper containing peptides, vitamins such as vitamin E, vitamin A, vitamin C, and vitamin B and vitamin salts or derivatives such as ascorbic acid di-glucoside and vitamin E acetate or palmitate; alpha hydroxy acids and their precursors such as glycolic acid, citric acid, lactic acid, malic acid, mandelic acid, ascorbic acid, alpha-hydroxybutyric acid, alpha-hydroxyisobutyric acid, alpha-hydroxyisocaproic acid, atrrolactic acid, alpha-hydroxyisovaleric acid, ethyl pyruvate, galacturonic acid, glucoheptonic acid, glucoheptono 1,4-lactone, gluconic acid, gluconolactone, glucuronic acid, glucuronolactone, isopropyl pyruvate, methyl pyruvate, mucic acid, pyruvic acid, saccharic acid, saccaric acid 1,4-lactone, tartaric acid, and tartronic acid; beta hydroxy acids such as beta-hydroxybutyric acid, beta-phenyl-lactic acid, and beta-phenylpyruvic acid; tetrahydroxypropyl ethylene-diamine, N,N,N′,N′-Tetrakis(2-hydroxypropyl) ethylenediamine (THPED); and botanical extracts such as green tea, soy, milk thistle, algae, aloe, angelica, bitter orange, coffee, goldthread, grapefruit, hoellen, honeysuckle, Job's tears, lithospermum, mulberry, peony, puerarua, nice, and safflower; and salts and prodrugs thereof

### Topical Depigmentation Products

In one embodiment, the topical composition contains a depigmentation agent. Examples of suitable depigmentation agents include, but are not limited to: soy extract; soy isoflavones; retinoids such as retinol; kojic acid; kojic dipalmitate; hydroquinone; arbutin; transexamic acid; vitamins such as niacin and vitamin C; azelaic acid; linolenic acid and linoleic acid; placertia; licorice; and extracts such as chamomile and green tea; and salts and prodrugs thereof.

### Topical Antipsoriatic Products

In one embodiment, the topical composition contains an antipsoriatic active agent. Examples of antipsoriatic active agents (e.g., for seborrheic dermatitis treatment) include, but are not limited to, corticosteroids (e.g., betamethasone dipropionate, betamethasone valerate, clobetasol propionate, diflorasone diacetate, halobetasol propionate, triamcinonide, dexamethasone, fluocinonide, fluocinolone acetonide, halcinonide, triamcinolone acetate, hydrocortisone, hydrocortisone verlerate, hydrocortisone butyrate, aclometasone dipropionte, flurandrenolide, mometasone furoate, methylprednisolone acetate), methotrexate, cyclosporine, calcipotriene, anthraline, shale oil and derivatives thereof, elubiol, ketoconazole, coal tar, salicylic acid, zinc pyrithione, selenium sulfide, hydrocortisone, sulfur, menthol, and pramoxine hydrochloride, and salts and prodrugs thereof.

### Other Ingredients

In one embodiment, the topical composition contains a plant extract as an active agent. Examples of plant extracts include, but are not limited to, feverfew, soy, glycine soja, oatmeal, what, aloe vera, cranberry, witch-hazel, alnus, arnica, artemisia capillaris, asiasarum root, birch, calendula, chamomile, cnidium, comfrey, fennel, galla rhois, hawthorn, houttuynia, hypericum, jujube, kiwi, licorice, magnolia, olive, peppermint, philodendron, salvia, sasa albo- marginata, natural isoflavonoids, soy isoflavones, and natural essential oils.

In one embodiment, the topical composition contains a buffering agent such as citrate buffer, phosphate buffer, lactate buffer, gluconate buffer, or gelling agents, thickeners, or polymers.

In one embodiment, the topical composition contains a fragrance effective for reducing stress, calming, and/or affecting sleep such as lavender and chamomile.

### Topical Mucosal Products

In one embodiment, the topical composition is suitable for administering to a mucosal membrane, such as human oral, rectal, and vaginal musocal membranes. The topical compositions may be made into a wide variety of products for application on mucosa, including but not limited to vaginal creams, tampons, suppositories, floss, mouthwash, toothpaste. Other product forms can be formulated by those of ordinary skill in the art.

In one embodiment, the topical composition is used for the treatment of a mucosal membrane conditions. Examples of such treatments include, but are not limited to, treatment of vaginal candidiasis and bacterial vaginosis, genital and oral herpes, cold sore, canker sore, oral hygiene, periodontal disease, teeth whitening, halitosis, prevention of biofilm attachment, and other microbial infections of the mucosa.

The particulates can be incorporated into compositions for the treatment of candidiasis with actives such as, but not limited to: Tioconazole; Clotrimazole; and Nystatin. The particulates can be incorporated into compositions for the treatment of bacterial vaginosis with actives such as, but not limited to, Clindamycin Hydrochloride and Metronidazole.

The particulates can be incorporated into compositions for the treatment of periodontal disease with actives such as, but not limited to minocycline.

### Compositions for Treatment of Wounds and Scars

In one embodiment, the metal particulates are incorporated into wound dressings and bandages to provide electric therapy for healing enhancement and scar prevention. In one embodiment, the wound exudation fluid and/or wound cleansing solution serves to activate a particulate-containing wound dressing/bandage to (i) deliver active agents pre-incorporated in the wound dressing/bandage and/or (ii) to generate electrochemically beneficial metal ions followed with delivery of the beneficial metal ions into the wound, and/or (iii) treat the wound with therapeutic corrosion current which may increase blood circulation, stimulate tissue immune response, and/or suppress tissue inflammation, which may lead to accelerated healing and reduced scarring.

In one embodiment, the composition or product contains an active agent commonly used as for topical wound and scar treatment, such as topical antibiotics, anti-microbials, wound healing enhancing agents, topical antifungal drugs, anti-psoriatic drugs, and anti inflammatory agents.

Examples of antifungal drugs include but are not limited to miconazole, econazole, ketoconazole, sertaconazole, itraconazole, fluconazole, voriconazole, clioquinol, bifoconazole, terconazole, butoconazole, tioconazole, oxiconazole, sulconazole, saperconazole, clotrimazole, undecylenic acid, haloprogin, butenafine, tolnaftate, nystatin, ciclopirox olamine, terbinafine, amorolfine, naftifine, elubiol, griseofulvin, and their pharmaceutically acceptable salts and prodrugs. In one embodiment, the antifungal drug is an azole, an allylamine, or a mixture thereof.

Examples of antibiotics (or antiseptics) include but are not limited to mupirocin, neomycin sulfate bacitracin, polymyxin B, 1-ofloxacin, tetracyclines (chlortetracycline hydrochloride, oxytetracycline-10 hydrochloride and tetrachcycline hydrochoride), clindamycin phsphate, gentamicin sulfate, metronidazole, hexylresorcinol, methylbenzethonium chloride, phenol, quaternary ammonium compounds, tea tree oil, and their pharmaceutically acceptable salts and prodrugs.

Examples of antimicrobials include but are not limited to salts of chlorhexidine, such as lodopropynyl butylcarbamate, diazolidinyl urea, chlorhexidene digluconate, chlorhexidene acetate, chlorhexidene isethionate, and chlorhexidene hydrochloride. Other cationic antimicrobials may also be used, such as benzalkonium chloride, benzethonium chloride, triclocarbon, polyhexamethylene biguanide, cetylpyridium chloride, methyl and benzothonium chloride. Other antimicrobials include, but are not limited to: halogenated phenolic compounds, such as 2,4,4′,-trichloro-2-hydroxy diphenyl ether (Triclosan); parachlorometa xylenol (PCMX); and short chain alcohols, such as ethanol, propanol, and the like. In one embodiment, the alcohol is at a low concentration (e.g., less than about 10% by weight of the carrier, such as less than 5% by weight of the carrier) so that it does not cause undue drying of the barrier membrane.

Examples of anti-viral agents for viral infections such as herpes and hepatitis, include, but are not limited to, imiquimod and its derivatives, podofilox, podophyllin, interferon alpha, acyclovir, famcyclovir, valcyclovir, reticulos and cidofovir, and salts and prodrugs thereof

Examples of anti-inflammatory agent, include, but are not limited to, suitable steroidal anti-inflammatory agents such as corticosteroids such as hydrocortisone, hydroxyltriamcinolone alphamethyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amciafel, amcinafide, betamethasone, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone, and salts are prodrugs thereof In one embodiment, the steroidal anti-inflammatory for use in the present invention is hydrocortisone. A second class of anti-inflammatory agents which is useful in the compositions of the present invention includes the nonsteroidal anti inflammatory agents.

Examples of wound healing enhancing agent include recombinant human platelet-derived growth factor (PDGF) and other growth factors, ketanserin, iloprost, prostaglandin E1 and hyaluronic acid, scar reducing agents such as mannose-6-phosphate, analgesic agents, anesthetics, hair growth enhancing agents such as minoxadil, hair growth retarding agents such as eflornithine hydrochloride, antihypertensives, drugs to treat coronary artery diseases, anticancer agents, endocrine and metabolic medication, neurologic medications, medication for cessation of chemical additions, motion sickness, protein and peptide drugs.

### Treatment of Microbial Infections of the Body

In one embodiment, the particulates are used, with or without antifungal active agents, to treat and prevent fungal infections (e.g., dermatophytes such as trichophyton mentagrophytes), including, but not limited to, onychomycosis, sporotrichosis, tinea unguium, tinea pedis (athlete's foot), Tinea cruris (jock itch), tinea corporis (ringworm), tinea capitis, tinea versicolor, and Candida yeast infection-related diseases (e.g., Candida albicans) such as diaper rash, oral thrushm, cutaneous and vaginal candidiasis, genital rashes, Malassezia furfur infection-related diseases such as Pityriasis versicolor, Pityriasis folliculitis, seborrhoeic dermatitis, and dandruff.

In another embodiment, the particulates are used, with or without antibacterial active agents, to treat and prevent bacterial infections, including, but not limited to, acne, cellulitis, erysipelas, impetigo, folliculitis, and furuncles and carbuncles, as well as acute wounds and chronic wounds (venous ulcers, diabetic ulcers and pressure ulcers).

In another embodiment, the particulates are used, with or without antiviral active agents, to treat and prevent viral infections of the skin and mucosa, including, but not limited to, molluscum contagiosum, warts, herpes simplex virus infections such as cold sores, kanker sores and genital herpes.

In another embodiment, the particulates are used, with or without antiparasitic active agents, to treat and prevent parasitic infections, including, but not limited to, hookworm infection, lice, scabies, sea bathers' eruption and swimmer's itch.

In one embodiment, the particulates are administered to help treat ear infections (such as those caused by streptococcus pneumoniae), rhinitis and/or sinusitis (such as caused by Haemophilus influenzae, Moraxella catarrhalis, Staphylococcus aureus and Streptococcus pneumoniae), and strep throat (such as caused by Streptococcus pyogenes).

In one embodiment, the particulates are ingested by an animal (e.g., as animal feed) or a human (e.g., as a dietary supplement) to help prevent outbreaks of food borne illnesses (e.g., stemming from food borne pathogens such as Campylobacter jejuni, Listeria monocytogenes, and Salmonella enterica).

### Treatment of Drug Resistant Microorganisms

In one embodiment, the invention features a method of killing pathogens drug resistant microorganisms by contacting the microorganism with a composition containing a metal particulate of the invention. In one embodiment, the drug resistant microorganism is a bacterium, such as MRSA and VRE. In one embodiment, the particulates are administered via a nasal spray, rinse solution, or ointment.

### Nail Treatment Products

The particulates can also be used to stimulate nail growth, enhance nail strength, and reduce nail infection or discoloration. The particulates can be incorporated into compositions for the treatment of onychomychosis with actives such as, but not limited to: miconazole, econazole, ketoconazole, sertaconazole, itraconazole, fluconazole, voriconazole, clioquinol, bifoconazole, terconazole, butoconazole, tioconazole, oxiconazole, sulconazole, saperconazole, clotrimazole, undecylenic acid, haloprogin, butenafine, tolnaftate, nystatin, ciclopirox olamine, terbinafine, amorolfine, naftifine, elubiol, griseofulvin, and their pharmaceutically acceptable salts and prodrugs. Particulates can be incorporated into compositions for improving the look and feel of nails with ingredients such as, but not limited to: biotin, calcium panthotenate, tocopheryl acetate, panthenol, phytantriol, cholecalciferol, calcium chloride, Aloe Barbadensis (Leaf Juice), silk Protein, soy protein, hydrogen peroxide, carbamide peroxide, green tea extract, acetylcysteine and cysteine.

### Tissue-Augmentation Products

In one embodiment, the particulates can be used to reduce the visibility of skin facial wrinkles, reduce atrophy, or increase collagen stimulation. The particulates may be used either alone or in conjunction with other components well known in the art, such as subcutaneous fillers, implants, periodontal implants, intramuscular injections, and subcutaneous injections, such as bio-absorbable polymers. For example, the particulates may be used in conjunction with collagen and/or hyaluronic acid injections.

In another embodiment, the particulates can be incorporated into biodegradable scaffolds for tissue engineering and organ printing with techniques known in the art.

### Transdermal Drug Delivery Patches

In one embodiment, the particulates are incorporated into transdermal drug delivery patches to enhance active agent penetration into the skin by iontophoresis and to reduce skin irritation by electric stimulation and electrically generated beneficial ions, such as zinc ions. Examples of such active agents include peptides, polypeptides, proteins, and nucleic acid materials comprising DNA; and nutrients. Examples of polypeptide and protein active agents include thyrotropin-releasing hormone (TRH), vasopressin, gonadotropin-releasing hormone (GnRH or LHRH), melanotropin-stimulating hormone (MSH), calcitonin, growth hormone releasing factor (GRF), insulin, erythropoietin (EPO), interferon alpha, interferon beta, oxytocin, captopril, bradykinin, atriopeptin, cholecystokinin, endorphins, nerve growth factor, melanocyte inhibitor-I, gastrin antagonist, somatotatin, encephalins, melatonin, vaccines, botox (Botulinum neurotoxins), cyclosporin and its derivatives (e.g., biologically active fragments or analogs). Other active agents include anesthetics; analgesics (e.g., fentanyl and salts thereof such fentanyl citrate); drugs for treating psychiatric disorders, epilepsies, and migraine; drugs for stopping drug additions and abuses; anti-inflammatory agents; drugs to treat hypertension, cardiovascular diseases, gastric acidity and ulcers; drugs for hormone replacement therapies and contraceptives such as estrogens and androgens; antibiotics, antifungals, antiviral and other antimicrobial agents; antineoplastic agents, immunosuppressive agents and immune-stimulants; and drugs acting on blood and the blood forming argans including hematopoietic agents and anticoagulants, thrombolytics, and antiplatelet drugs. Other active agents that can be delivered into the body using such patches include vaccines for various diseases, such as those for influenza, AIDS, hepatitis, measles, mumps, rubella, rabies, rubella, avercella, tetanus, hypogammaglobulinemia, Rh disease, diphtheria, botulism, snakebite, back widow bite and other insect bite/sting, idiopathic thrombocytopenic purpura (ITP), chronic lymphocytic leukemia, cytomegalovirus (CMV) infection, acute renal rejection, oral polio, tuberculosis, pertussis, Haemophilus b, Pneumococcus, and Staphylococcus aureus.

### Medical Surgical Treatments, Medical Implants and Surgical Products

In one embodiment, the particulates are incorporated into or onto a medical device or an implant. Suitable medical devices that may contain or be coated with the particulates include, but are not limited to, wound closure staples, sutures, suture anchors, surgical needles, hypodermic needles, catheters, wound tape, wound dressing, hemostats, stents, vascular grafts, vascular patches, catheters, surgical meshes, bone implants, joint implants, prosthetic implants, bone grafts, dental implants, breast implants, tissue augmentation implants, plastic reconstruction implants, implantable drug delivery pumps, diagnostic implants and tissue engineering scaffolds and other conventional medical devices and equivalents thereof. The medical devices may be prepared or made from conventional biocompatible absorbable or resorbable polymers, nonabsorbable polymers, metals, glasses or ceramics and equivalents thereof

Suitable nonabsorbable polymers include, but are not limited to acrylics, polyamide-imide (PAI), polyarcryletherketones (PEEK), polycarbonate, polyethylenes (PE), polybutylene terephthalates (PBT) and polyethylene(PET), terephthalates, polypropylene, polyamide (PA), polyvinylidene fluoride (PVDF), and polyvinylidene fluoride,-co-hexafluropropylene(PVDF/HFP), polymethylmetacrylate(PMMA) and combinations thereof and equivalents.

Suitable absorbable polymers may be synthetic or natural polymers. Suitable biocompatible, bioabsorbable polymers include polymers selected from the group consisting of aliphatic polyesters, poly (amino acids), copoly (ether-esters), polyalkylenes oxalates, polyamides, tyrosine derived polycarbonates, poly (iminocarbonates), polyorthoesters, polyoxaesters, polyamidoesters, polyoxaesters containing amine groups, poly (anhydrides), polyphosphazenes, and combinations thereof For the purpose of this invention aliphatic polyesters include, but are not limited to, homopolymers and copolymers of lactide (which includes lactic acid, D-, L- and meso lactide), glycolide (including glycolic acid), epsilon-caprolactone, p-dioxanone (1,4-dioxan-2-one), trimethylene carbonate (1,3-dioxan-2-one), alkyl derivatives of trimethylene carbonate, and polymer blends thereof Natural polymers include collagen, elastin, hyaluronic acid, laminin, and gelatin, keratin, chondroitin sulfate and decellularized tissue.

Suitable metals are those biocompatible metals used conventionally in medical devices including, but not limited to titanium, titanium alloys, tantalum, tantalum alloys, stainless steel, and cobalt-chromium alloys (e.g., cobalt-chromium-molybdenum alloy) and the like. These metals are conventionally used in sutures, surgical needles, orthopedic implants, wound staples, vascular staples, heart valves, plastic surgery implants, other implantable devices and the like.

Suitable absorbable or biocompatible glasses or ceramics include, but are not limited to phosphates such as hydroxyapatite, substituted apatites, tetracalcium phosphate, alpha-and beta-tricalcium phosphate, octacalcium phosphate, brushite, monetite, metaphosphates, pyrophosphates, phosphate glasses, carbonates, sulfates and oxides of calcium and magnesium, and combinations thereof.

In the practice of the present invention, particulates may be combined with medical devices by various methods including coating the particulate on at least part of a surface of the medical device, incorporating the particulate into the medical device, and combinations thereof Incorporating the particulate into the medical device allows for a sustained activity of the particulates which are exposed over time as in the case of absorbable polymers. The particulates are activated by moisture; therefore all processing of the particulates should be carried out under dry or substantially dry conditions.

The particulate may be coated on the surface of the medical device by directly attaching the particulates to the device or by using a polymeric binder, including conventional biocompatible polymeric binders. The particulates may also be directly attached to the device by heating the particulates. The particulates may be attached to the surface of a medical device prepared from polymers or devices having a polymer coating as a binder by heating the particulates to a temperature sufficient to melt the surface of the medical device, followed by impacting the particulate with the device surface, which temporarily melts or softens the surface and then cools allowing the particulate to be placed on or embedded in or otherwise adhered to the surface of the device. The heated particulates may be applied by conventional coating methods such as electrostatic spraying, fluidized bed coating, and the like. Alternatively, a polymeric film can be coated on the surface of a device, and this film is then heated and the particulates are applied to the softened film as described above.

Alternatively a polymer binder coating may be used to apply or attach the particulates to the medical devices. The particulates may be combined with a solution containing the polymer binder. Suitable polymer binders include those used to prepare medical devices listed above. Suitable solvents include 1,4-dioxane, ethyl acetate and the like. One of skill in the art can determine the appropriate solvent based upon the polymer composition. The polymer binder is dissolved in a suitable solvent in the concentration of about 1 weight % to about 15 weight %. The particulates may be present in the polymer binder solution in the amount of about 7.5 weight % to about 10 weight %. The coatings containing the particulates in the polymer binder solution may be used to coat the medical devices, typically all or part of outer surfaces although inner surfaces may be coated as well, by conventional methods such as microspray coating, electrostatic spraying, electrostatic spinning, dip coating, fluidized bed coating and the like.

In one embodiment, the amount of particulates on the coated surface of a medical device is sufficient to elicit antimicrobial and/or anti-inflammatory and/or anti-adhesion effects in a safe and efficacious manner. In one embodiment, the particulates may be present on the surface of the device in the amount of about 0.001 mg/in² to about 20 mg/in². In another embodiment the particulates may be present on the surface of the device in the amount of about 0.1 mg/in² to 10 mg/in².

The particulates may also be incorporated into the medical device by conventional methods such as compounding, solvent casting, lyophilization, electrostatic spinning, extrusion, and the like.

The particulates may be compounded into a composite with molten polymers in a static mixer or continuous extruder. The composite of the particulates and polymer can be further processed into devices using methods including extrusion, injection molding, compression molding, and other melting processes. Suitable polymers include those used to prepare medical devices listed above. In one embodiment, the particulate loading in the composite may be about 0.001 weight % to about 80% by weight. In another embodiment, the particulate loading in the composite may be about 0.01 weight % to about 20 weight %. One of skill in the art can determine suitable processing conditions for the desired polymer composition.

Alternatively, a polymer solution may be used to incorporate the particulates into the medical devices by methods such as solvent casting, lyophilization, electrostatic spinning and the like. The particulates may be combined with a polymer solution. Suitable polymers include those used to prepare medical devices listed above. Suitable solvents include 1,4-dioxane, ethyl acetate and the like. One of skill in the art can determine the appropriate solvent based upon the polymer composition. The polymer is dissolved in a suitable solvent in the concentration of about 1 weight % to about 15 weight %. The particulates may be present in the polymer solution in the amount of about 7.5 weight% to about 10 weight %. Such particulate/polymer solutions may be used in conventional processes including solvent casting to provide films, lyophilization to provide foam medical devices, and electrostatic spinning to prepare fibers, tubes, mats and the like.

The particulates may also be combined with an aqueous composition, such as aqueous gel or emulsion. The particulates may be mixed with an aqueous gel at the point of use. The particulates may be present in the aqueous gel in the amount of about 0.001 weight % to about 10 weight %, and preferably about 0.01 weight % to about 1 weight %. In another embodiment, a mixture of particulates and suitable polymers in a dry form may be hydrated at the point of use. The suitable polymers include, but are not limited to carboxyl methylcellulose, hyaluronic acid, PEG, alginate, chitosan, chondroitin sulfate, dextran sulfate, and polymer blend and their salts thereof. Suitable aqueous solvents are water, physiological saline, phosphate-buffered saline, and the like.

Medical devices of the present invention comprising particulates are useful for preventing, reducing or eliminating infection at the implant site. It will be appreciated that such devices will be used with other aspects of infection control including sterile procedures, antibiotic administration, etc. For example, mesh coated with particulates (or otherwise containing particulates) can be used for contaminated hernia repair or contaminated trauma repair with significantly reduced concerns about the generation of anti-biotic resistant bacteria including biofilms. Alternatively, an anti-infective hemostat containing particulates can be useful for traumatic and post-surgical bleeding control. The medical devices of the present invention having particulates can be used in addition to conventional methods for infection control, such as oral or IV administration of antibiotics to enhance the efficacy of the conventional treatment methods for infection control. Incorporation in and coating of medical devices with particulates can improve the biocompatibility of the devices and enhance tissue-device integration and promote wound repair by suppressing inflammatory reaction.

In one embodiment, the medical devices with particulates are used to provide the intended therapeutic stimulation effects via corrosion current to promote tissue regeneration, repair and growth by applying the particulates directly to the target location of the body in need such a therapeutic treatment (e.g., either topically or inside the body), including soft tissues, hard tissues, and soft tissue-hard tissue conjunctions. In one embodiment, the medical device comprising particulates is administered alone. In another embodiment, additional particulates are administered locally, but separately and in a different dosage form from the particulate-containing medical device. One non-limiting example is a particulate-containing gel composition administered to the surgical site where a particulate-coated medical implant is implanted.

Such therapeutic effects include, but are not limited to: antimicrobial effects (e.g., antibacterial, antifungal, antiviral, and anti-parasitic effects); anti-inflammation effects including effects in the superficial or deep tissues (e.g., reduce or elimination of soft tissue edema or redness); prevention of post-surgical tissue adhesion (anti-adhesion); elimination or reduction of pain, itch or other sensory discomfort (e.g., headache, sting or tingling numbness); regeneration or healing enhancement of both soft and hard tissues; modulation of stem cell differentiation and tissue development such as modulation of tissue growth (e.g., enhancing growth rate of the nail or regrowth of hair loss due to alopecia) or increase soft tissue volume (e.g., increasing collagen or elastin in the skin or lips); increasing adepocyte metabolism or improving body appearance (e.g., effects on body contour or shape); and increasing circulation of blood or lymphocytes.

In one embodiment, the medical devices comprising particulates provide multiple mechanisms of actions to treat conditions, such as by enhancing delivery of an active agent by iontophoresis and/or electro-osmosis as well as by providing electric stimulation (e.g., to increase blood circulation or other benefits).

In one embodiment, the medical devices with particulates can be combined with an active agent (such as antimicrobial agents, anti-inflammatory agents, analgesic agents, and biological agents) incorporated into a medical device (e.g., as a surface coating or embedded within) to enhance or potentiate the biological or therapeutic effects of that active agent. In another embodiment, the particulates can be incorporated into a medical device to work efficacious or synergistically with one or more than one active agent administered by a different route of administration concurrently or sequentially (e.g., by systemic route such as oral dosing, injection or infusion) to enhance or potentiate the biological or therapeutic effects of that active agent. For example, a medical implant with a particulate coating can be applied to a patient through a surgical procedure, whereas a systemic antibiotic therapy can be administered either prior to or shortly after the procedure as prophylaxis to prevent or treat any post-surgical infections. In yet another embodiment, the particulates can also be combined with other substances to enhance or potentiate the activity of the particulates. Substances that can enhance or potentiate the activity of the particulates include, but are not limited to, organic solvents, surfactants, and water-soluble polymers. For example, the particulates of the present invention can form conjugates or composites with synthetic or natural polymers including by not limited to proteins, polysaccharides, hyaluronic acid of various molecular weight, hyaluronic acid analogs, polypeptides, and collagens of different origins.

In one embodiment, the composition contains a chelator or chelating agent. Examples of chelators include, but are not limited to, amino acids such as glycine, lactoferrin, edetate, citrate, pentetate, tromethamine, sorbate, ascorbate, deferoxamine, derivatives thereof, and mixtures thereof. Other examples of chelators useful are disclosed in U.S. Pat. No. 5,487,884 and PCT Publication No. WO2006056984.

In one embodiment, the composition or product contains an active agent commonly used as for topical wound and scar treatment (such as topical antibiotics, anti-microbials, wound healing enhancing agents, topical antifungal drugs, anti-psoriatic drugs, and anti-inflammatory agents), but can also be used for internal (inside body) applications. Such active agents have been disclosed in the previous sections of the present invention.

Examples of antifungal drugs include but are not limited to miconazole, econazole, ketoconazole, sertaconazole, itraconazole, fluconazole, voriconazole, clioquinol, bifoconazole, terconazole, butoconazole, tioconazole, oxiconazole, sulconazole, saperconazole, clotrimazole, undecylenic acid, haloprogin, butenafine, tolnaftate, nystatin, ciclopirox olamine, terbinafine, amorolfine, naftifine, elubiol, griseofulvin, and their pharmaceutically acceptable salts and prodrugs. In one embodiment, the antifungal drug is an azole, an allylamine, or a mixture thereof.

Examples of antibiotics (or antiseptics) include but are not limited to mupirocin, neomycin sulfate bacitracin, polymyxin B, 1-ofloxacin, tetracyclines (chlortetracycline hydrochloride, oxytetracycline-10 hydrochloride and tetrachcycline hydrochoride), clindamycin phsphate, gentamicin sulfate, metronidazole, hexylresorcinol, methylbenzethonium chloride, phenol, quaternary ammonium compounds, tea tree oil, and their pharmaceutically acceptable salts and prodrugs.

Examples of antimicrobials include but are not limited to octenidine, salts of chlorhexidine, such as lodopropynyl butylcarbamate, diazolidinyl urea, chlorhexidene digluconate, chlorhexidene acetate, chlorhexidene isethionate, and chlorhexidene hydrochloride. Other cationic antimicrobials may also be used, such as benzalkonium chloride, benzethonium chloride, triclocarbon, polyhexamethylene biguanide, cetylpyridium chloride, methyl and benzothonium chloride. Other antimicrobials include, but are not limited to halogenated phenolic compounds, such as 2,4,4′,-trichloro-2-hydroxy diphenyl ether (Triclosan); parachlorometa xylenol (PCMX); and short chain alcohols, such as ethanol, propanol, and the like.

Examples of anti-viral agents for viral infections such as herpes and hepatitis, include, but are not limited to, imiquimod and its derivatives, podofilox, podophyllin, interferon alpha, acyclovir, famcyclovir, valcyclovir, reticulos and cidofovir, and salts and prodrugs thereof.

Examples of anti-inflammatory agents, include, but are not limited to, suitable steroidal anti-inflammatory agents such as corticosteroids such as hydrocortisone, hydroxyltriamcinolone alphamethyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amciafel, amcinafide, betamethasone, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone, and salts are prodrugs thereof In one embodiment, the steroidal anti-inflammatory for use in the present invention is hydrocortisone. A second class of anti-inflammatory agents which is useful in the compositions of the present invention includes the nonsteroidal anti-inflammatory agents.

Examples of wound healing enhancing agents include recombinant human platelet-derived growth factor (PDGF) and other growth factors, ketanserin, iloprost, prostaglandin E₁and hyaluronic acid, scar reducing agents such as mannose-6-phosphate, analgesic agents, anesthetics, hair growth enhancing agents such as minoxadil, hair growth retarding agents such as eflornithine hydrochloride, antihypertensives, drugs to treat coronary artery diseases, anticancer agents, endocrine and metabolic medication, neurologic medications, medication for cessation of chemical additions, motion sickness, protein and peptide drugs.

In one embodiment, the particulates are used, with or without antifungal active agents, to treat and prevent fungal infections. In another embodiment, the particulates are used, with or without other antibacterial active agents, to treat and prevent bacterial infections, including, but not limited to, infections of tissue injuries of intern or surface of the body due to surgical procedures such as acute wounds, and chronic wounds due to various illnesses (venous ulcers, diabetic ulcers and pressure ulcers).

In another embodiment, the particulates are used, with or without antiviral active agents, to treat and prevent viral infections of the skin and mucosa, including, but not limited to, molluscum contagiosum, warts, herpes simplex virus infections such as cold sores, canker sores and genital herpes.

In another embodiment, the particulates are used, with or without antiparasitic active agents, to treat and prevent parasitic infections, including, but not limited to, hookworm infection, lice, scabies, sea bathers' eruption and swimmer's itch.

In another embodiment, the particulates can be incorporated into biodegradable scaffolds for tissue engineering and organ printing with techniques known in the art.

In another embodiment, the particulates can be incorporated into aqueous gels for tissue adhesion prevention. For example, particulates in carboxyl methylcellulose aqueous solution or gel may be applied to a trauma site and surrounding tissue to reduce adhesion scar formation.

In another embodiment, the particulates can be incorporated into aqueous gels for osteoarthritis treatment to eliminate or reduce pain via intra-articular injection. For example, the particulates may be contained in a hyaluronic acid-containing gel and applied, preferably injected, into an articulating joint (shoulder, elbow, ankle, carpometacarpal (CMC), i.e., thumb joint, or hip) suffering from arthritis.

In another embodiment, the particulates can be incorporated into an aqueous gel or an anhydrous gel for wound treatment to eliminate or reduce pain caused by inflammation, and to prevent or treat infection, to enhance healing rate and/or strength, and to reduce scarring.

The particulates may also be combined with an aqueous composition, such as aqueous gels or emulsions. The particulates may be mixed with an aqueous gel at the point of use. The particulates may be present in the aqueous gel in the amount of about 0.01 weight % to about 0.5 weight %, and preferably about 0.05 weight % to about 0.25weight %. In another embodiment, a mixture of particulates and suitable polymers in a dry form may be hydrated at the point of use. The suitable polymers include, but are not limited to carboxyl methylcellulose, hyaluronic acid, PEG, alginate, chitosan, chondroitin sulfate, dextran sulfate, and polymer blend and their salts thereof. Suitable aqueous solvents are water, physiological saline, phosphate-buffered saline, and the like. In another embodiment, the polymer(s) as gelling agent may be present in the aqueous gel in the amount of about 0.01 weight % to about 20 weight %, and preferably about 0.1 weight % to about 5 weight %.

In another embodiment, the particulates can be incorporated to the surface coating of a breast implant to improve the biocompatibility of implants and provide anti-microbial and anti-inflammatory benefits to eliminate or reduce capsular contracture.

In another embodiment, the medical devices of the present invention comprising particulates can be used with other energy-based medical devices and treatments to increase the therapeutic efficacy of either or both devices. The energy-based treatments include, but are not limited to, ultrasound device or therapy, magnetic treatment, electromagnetic device or therapy, radiofrequency treatment, thermal treatment (heating or cooling).

The novel medical devices of the present invention containing particulates can be used in various conventional surgical procedures, including but not limited to open and minimally invasive surgical procedures, for implanting medical devices and other implants such as wound closure following a surgical procedure, wound closure of traumatic injuries, catheter insertion, application of hemostats, stent implantation, insertion of vascular grafts and vascular patches, implanting surgical meshes, implanting bone implants, orthopedic implants and soft tissue implants, implanting bone grafts and dental implants, cosmetic surgery procedures, including implanting breast implants, tissue augmentation implants, and plastic reconstruction implants, inserting drug delivery pumps, inserting or implanting diagnostic implants, implanting tissue engineering scaffolds, and other surgical procedures requiring long term or permanent implants. The medical devices are implanted using surgical procedures in a conventional manner to obtain the desired result, and in addition, the use of the novel devices of the present invention provides for improved surgical outcomes by reducing infection and biofilm formation, suppressing inflammation and enhancing tissue repair and regeneration.

### Treatment of Joint Conditions

In one aspect, compositions are disclosed for treating a joint condition. The composition can be in liquid form. The liquid composition can also be stable at room temperature. Moreover, the liquid composition can include a solution of hyaluronic acid (HA) along with the particulates of the invention. The composition can comprise a high molecular weight HA. The molecular weight can be, for example, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000 kDa or more, or any range derivable therein. In exemplary embodiments, the HA has a molecular weight in the range of about 1 MDa to 6 MDa. In another exemplary embodiment, the HA has a molecular weight greater than 1 MDa.

Moreover, the HA can be present at particular concentrations. In one embodiment, the HA is present at a concentration of at least about 7 mg/ml. In another exemplary embodiment, the HA has a concentration of at least about 5 mg/ml, and more preferably at least about 7 mg/ml, and more preferably at least about 10 mg/ml, and more preferably at least about 15 mg/ml, and in some embodiments the concentration can be at least about 25 mg/ml. In another embodiment, the HA can have a concentration in the range of about 15 mg/ml to about 25 mg/ml.

In another aspect, such composition includes at least one additional component. The additional component can be selected from, for example, amino acids, amino sugars, sugar alcohols, proteins, saccharides, di-saccharides, oligo-saccharides, poly-saccharides, nucleic acids, buffers, surfactants, lipids, liposomes, other excipients, and mixtures thereof Other useful components can include steroids, anti-inflammatory agents, non-steroidal anti-inflammatory agents, analgesics, cells, antibiotics, antimicrobial agents, anti-inflammatory agents, growth factors, growth factor fragments, small-molecule wound healing stimulants, hormones, cytokines, peptides, antibodies, enzymes, isolated cells, platelets, immunosuppressants, nucleic acids, cell types, viruses, virus particles, essential nutrients, minerals, metals, or vitamins, and combinations thereof Additionally, the formulation or composition can include a diluent, such as water, saline, or a buffer.

Hyaluronic acid (HA) can have various formulations and can be provided at various concentrations and molecular weights. The terms “hyaluronic acid,” “hyaluronan,” “hyaluronate,” and “HA” are used interchangeably herein to refer to hyaluronic acids or salts of hyaluronic acid, such as the sodium, potassium, magnesium, and calcium salts, among others. These terms are also intended to include not only pure hyaluronic acid solutions, but hyaluronic acid with other trace elements or in various compositions with other elements. The terms “hyaluronic acid,” “hyaluronan,” and “HA” encompass chemical or polymeric or cross-linked derivatives of HA. Examples of chemical modifications which may be made to HA include any reaction of an agent with the four reactive groups of HA, namely the acetamido, carboxyl, hydroxyl, and the reducing end. The HA used in the present application is intended to include natural formulations (isolated from animal tissue) or synthetic formulations (derived from bacterial fermentation) or combinations thereof The HA can be provided in liquid form or solid formulations that is reconstituted with a diluents to achieve an appropriate concentration.

The methods of treatment can include directly injecting the compositions into the target area, such as a joint. Injections can be performed as often as daily, weekly, several times a week, bi monthly, several times a month, monthly, or as often as needed as to provide relief of symptoms. For intra-articular use, from about 1 to about 30 mg/ml of HA per joint, depending on the size of the joint and severity of the condition, can be injected. The frequency of subsequent injections into a given joint are spaced to the time of recurrence of symptoms in the joint. Illustratively, dosage levels in humans of the composition can be: knee, about 1 to about 30 mg/ml per joint injection; shoulder, about 1 to about 30 mg/ml of HA per joint injection; metacorpal or proximal intraphalangeal, about 1 mg/ml to about 30 mg/ml of HA per joint injection; and elbow, about 1 to about 30 mg/ml per joint injection. The total amount of injection can range from about 1 mg/ml to 200 mg/ml of HA.

It will be understood, however, that the specific dosage level for any particular patient will depend upon a variety of factors including the activity of the specific compound employed, the age, body weight, general health, gender, diet, time of administration, route of administration, rate of excretion, drug combination and the severity of the particular disease undergoing therapy. The compositions can be prepared and administered in dose units. Under certain circumstances, however, higher or lower dose units may be appropriate. The administration of the dose unit can be carried out both by single administration of the composition or administration can be performed in several smaller dose units and also by multiple administrations of subdivided doses at specific intervals.

In one embodiment, the medical condition is osteoarthritis (OA) and the composition is administered in a joint space, such as, for example, a knee, shoulder, temporo-mandibular and carpo-metacarpal joints, elbow, hip, wrist, ankle, and lumbar zygapophysial (facet) joints in the spine. The viscosupplementation may be accomplished via a single injection or multiple intra-articular injections administered over a period of weeks into the knee or other afflicted joints. For example, a human subject with knee OA may receive one, two, three, four, or five injections of about 0.5, 1, 1.5, 2, 3, 4, 5, 6, 7, 8, 9, 10 ml or more per knee. For other joints, the administered volume can be adjusted based on the size on the joint.

### Prevention of Post-Surgical Tissue Adhesion

Adhesion formation, in particular following peritoneal, thoracic, and spinal surgery, for example, is a major source of postoperative morbidity and mortality. Appendectomy and gynecologic surgery, for example, are the most frequent surgical procedures implicated in clinically significant adhesion formation. The most serious complication of intraperitoneal adhesions is intestinal obstruction. In addition, adhesions are associated with chronic or recurrent pelvic pain and infertility in females, nerve compression and pain in the spine, post-operative complications following thoracic surgery, and loss of mobility in the hand after reconstructive surgery.

The invention also provides treatments to inhibit or prevent the formation of post-operative adhesions, as well as compositions for use in such treatments utilizing nutritional components. Such compositions comprise metal particulates in a biocompatible delivery vehicle that may be delivered directly to the surgical site to inhibit or prevent the formation of such adhesions.

The following examples are illustrative of the principles and practice of this invention, although not limited thereto. Numerous additional embodiments within the scope and spirit of the invention will become apparent to those skilled in the art once having the benefit of this disclosure.

### Example 1

Metal particulates comprising magnesium powder were obtained from Reade Manufacturing Company, Manchester, N.J. (RMC-325) and used to generate beneficial, low levels of hydrogen peroxide in human keratinocyte cells as follows.

The cells were seeded into an assay plate at identical densities and incubated for 48 hours at 37° C. with 5% CO₂ To detect hydrogen peroxide production, the keratinocytes were loaded for a 30-minute incubation period with 5 μM of the hydrogen peroxide-sensitive fluorescent probe 5-(and-6)-chloromethyl-2′,7′-dichlorodihydrofluorescein diacetate, acetyl ester (CM-H2DCFDA, Invitrogen Carlsbad, Calif.).

The cells were treated with different amounts of the magnesium powder. Treatment of control wells with 0.03% hydrogen peroxide served as a positive control. Hydrogen peroxide production was quantitated using a fluorescent plate reader set at wavelengths 485 nm excitation/530 nm emission. The test results are shown in Table 1.

TABLE 1

| Compound | 60 Minutes |
|---|---|
| Untreated | 37.54 ± 0.5 |
| Magnesium powder (10 mg/ml) | 199.1 ± 4.3** |
| Magnesium powder (5 mg/ml) | 191.9 ± 6.9** |
| Magnesium powder (1 mg/ml) | 50.58 ± 0.8** |
| H₂O₂ (0.03%) | 150.11 ± 2.9** |

**Indicates significant difference from untreated group using a student's t-Test with significance set at P < 0.05.

The data in Table 1 shows that particulates generated low levels of hydrogen peroxide in human keratinocyte cells in situ comparable to adding 0.03 weight percent hydrogen peroxide directly to the cells. Applicants have discovered that such low levels of in situ generated hydrogen peroxide provide anti-inflammation benefits to a variety of tissues.

### Example 2

The effect of topical application of particulates comprising magnesium or zinc for anti-inflammatory activity on human epidermal equivalents was evaluated as follows.

Particulates comprising zinc powder were obtained from U.S. ZINC, Houston, Tex. (USZ-1 XLP).

Particulates comprising magnesium powder were obtained from Reade Manufacturing Company, Manchester, N.J. (RMC-325).

Epidermal equivalents (EPI 200 HCF), multilayer and differentiated epidermis consisting of normal human epidermal keratinocytes, were obtained from MatTek (Ashland, Mass.). Upon receipt, the epidermal equivalents were incubated for 24 hours at 37° C. in maintenance medium without hydrocortisone. The equivalents were topically treated (2 mg/cm²) with the magnesium powder or the zinc powder (10 mg/ml) in a 70% ethanol/30% propylene glycol vehicle 2 hours before exposure to solar ultraviolet light (1000W-Oriel solar simulator equipped with a 1-mm Schott WG 320 filter; UV dose applied: 70 kJ/m² as measured at 360 nm). The equivalents were incubated for 24 hours at 37° C. with a maintenance medium, and then the supernatants were analyzed for IL-8 cytokine release using commercially available kits (Upstate Biotechnology, Charlottesville, Va.). The test results are shown in Table 2.

TABLE 2

| | Mean +/− Std | |
|---|---|---|
| | Dev of IL-1A | Percent Inhibition of |
| Treatment (Dose, as % w/v) | Release (ng/ml) | Skin Inflammation |
| Untreated, No UV | 1.18 ± 0.5 | — |
| UV (60 KJ), Vehicle Treated | 999.5 ± 148.2 | — |
| UV (60 KJ) + Zinc | 414.6 ± 112.2** | 58.5% |
| powder (10 mg/ml) | | |
| Untreated, No UV | 69.38 ± 8.3 | — |
| UV (60 KJ), Vehicle Treated | 695.8 ± 71.2 | — |
| UV (60 KJ) + Magnesium | 301.5 ± 82.2** | 57.0% |
| powder (10 mg/ml) | | |

**Indicates significant difference from UV, Vehicle treated using a student's t-Test with significance set at P < 0.05.

The data in Table 2 shows that the topical application of particulates comprising magnesium or zinc significantly reduced the UV-stimulated release of inflammatory mediators.

### Example 3

The ability of topically applied particulates comprising magnesium or zinc as described above to affect the inflammatory response was demonstrated using in an in vivo immune cell-mediated skin inflammation model.

Albino male CD-1 mice, 7-9 weeks old, were induced on the shaved abdomen with 50 μl of 3% oxazolone in acetone/corn oil (Day 0). On Day 5, a 20 μl volume of 2% oxazolone in acetone was applied to the dorsal left ear of the mouse. Zinc powder or magnesium powder was then applied to the left ear in a volume of 20 μl. one hour after oxazolone challenge in a 70% ethanol/30% propylene glycol vehicle. The right ear was not treated. The mice were sacrificed by CO₂ inhalation 24 hours after the oxazolone challenge, the left and right ears were removed and a 7-mm biopsy was taken from each ear and weighed. The difference in biopsy weights between the right and left ear was calculated.

Anti-inflammatory effects were determined as an inhibition of the increase in ear weight. Application of 1 mg/ml of hydrocortisone, a known anti-inflammatory compound, was used as a positive control. The results are shown in Table 3, where the Percent Inhibition of Skin Inflammation was calculated as (Vehicle treated biopsy weight−Agent(s) treated biopsy weight)/(Vehicle treated biopsy weight)×100.

TABLE 3

| | Percent Inhibition |
|---|---|
| Treatment (Dose) | of Skin Inflammation* |
| Hydrocortisone (1 mg/ml) | 70.3% ± 6.6% |
| Magnesium powder (10 mg/ml) | 43.9% ± 12.9% |
| Zinc powder (10 mg/ml) | 47.5% ± 11.7% |

*% Inhibition = (Vehicle treated biopsy weight − Agent(s) treated biopsy weight)/(Vehicle treated biopsy weight) × 100

The above data shows that topical application of particulates demonstrated anti-inflammatory activity similar to a corticosteroid in a model of skin inflammation.

### Example 4

Metal particulates (zinc and magnesium powder) were tested for antimicrobial activity using a BacT/ALERT 3D Signature Model BTA 3D (Control Module Serial # 110CM1682, Incubator Serial # 109IL1760), Nuaire Biological safety cabinet, Model# NU-5437-500, Serial#86106091203 and a Lab-Line Incubator, Serial # 108903827-00.

The designated BacT/ALERT sample bottles containing the test microorganism and powder suspensions were loaded into the BacT/ALERT system where they were continuously agitated and automatically monitored for growth. The BacT/ALERT incubation temperature varied from 33-37° C., depending on the optimum growth requirements of the test microorganisms. The incubation time was set for 7-days, if no growth was detected the sample was flagged as negative for growth. For designated negative samples, when incubations were completed, 1-mL aliquots were pour plated in duplicate with molten TSA and/or subcultured into new BacT/ALERT sample bottles to differentiate between bacteriostatic versus bactericidal activity. Appropriate positive and negative process controls were included for each sample set.

The results are shown in Tables 4 and 5 below.

TABLE 4
BacT/ALERT Magnesium (Mg) Powder Antimicrobial Activity Results

| | | BacT/ALERT | | | |
|---|---|---|---|---|---|
| | Approx. | Time-to-Detection | | | |
| | Challenge | (Up to 7 Days) for | | | |
| | Population | designated Mg | | | Anti- |
| Test | Counts | powder suspensions (n = 2) | | | microbial |
| Microorganism | (CFU/mL) | 0.0% | 0.25% | 0.1% | Activity |
| Staphylococcus | 3.0 × 10⁵ | 0.38 days | Neg. | —* | Bacteri- |
| aureus | | | | | cidal |
| ATCC 65438 | | | | | |
| Pseudomonas | 1.4 × 10⁶ | 0.44 days | Neg. | Neg. | Bacteri- |
| aeruginosa | | | | | cidal |
| ATCC 9027 | | | | | |
| Candida albicans | 1.8 × 10⁴ | — | — | Neg. | — |
| ATCC 10231 | | | | | |
| Aspergillus | 1 × 10³ | — | — | Neg. | — |
| brasiliensis | | | | | |
| ATCC 16404 | | | | | |
| Staphylococcus | 1.9 × 10⁶ | 0.38 days** | — | Neg. | — |
| pseudintermedius | | | | | |
| (animal isolates) | | | | | |
| #1, #2 | | | | | |
| Pseudomonas | 1.0 × 10⁶ | 0.18 days** | — | Neg. | — |
| aeruginosa | | | | | |
| (animal isolates) | | | | | |
| #1, #2 | | | | | |
| Methicillin- | 1.9 × 10⁶ | 0.32 days** | — | Neg. | — |
| Resistant | | | | (n = | |
| Staphylococcus | | | | 3) | |
| pseudintermedius | | | | | |
| (animal isolates) | | | | | |
| #1, #2, #3 | | | | | |

*(not determined)
**(n = 1 sample size determined in a previous experiment for animal isolates designated #2 for each microbial species)

TABLE 5
BacT/ALERT Zinc Powder Antimicrobial Results

| | | BacT/ALERT | | |
|---|---|---|---|---|
| | | Time-to- | | |
| | | Detection | | |
| | | for designated | | |
| | Approximate | Zn Powder | | |
| | Concentration | suspension | | Antimicrobial |
| Test Microorganism | CFU/mL | 0.0% | 1.0% | Activity |
| Propionibacterium | 1.0 × 10⁶ | 0.56 days | Neg. | Bacteriostatic |
| acnes | 1.0 × 10² | 4.2 days | Neg. | Bactericidal |
| ATCC 6919 | | | | |
| Moraxella | 1.0 × 10⁶ | 0.34 days | Neg. | Bactericidal |
| catarrhalis | | | | |
| ATCC 8176 | | | | |
| Haemophilus | 1.0 × 10⁶ | 0.47 days | Neg. | Bactericidal |
| influenzae | | | | |
| ATCC 49247 | | | | |
| Streptococcus | 1.0 × 10⁶ | 0.85 days | Neg. | Bactericidal |
| pneumoniae | | | | |
| ATCC 49619 | | | | |
| Campylobacter | 1.0 × 10⁶ | 0.55 days | Neg. | Bactericidal |
| jejuni subsp. jejuni | | | | |
| ATCC 33291 | | | | |
| Streptococcus | 1.0 × 10⁶ | 0.38 days | Neg. | Bactericidal |
| pyogenes | | | | |
| ATCC 19615 | | | | |
| Group A | | | | |

### Example 5

### Part A: Preparation of Zinc Powder-HA Gel

A hyaluronic acid (HA) gel, sold under the tradename ORTHOVISC (Anika Therapeutics, Inc.) and distributed by DePuy Mitek, Inc. was used as a carrier. ORTHOVISC is a US FDA cleared, commercially available medical device for intra-articular injection for treatment of osteoarthritis pain. 20 mg of zinc powder/particulates as described in Example 2 were weighed and loaded into a sterile 3 ml syringe and capped. Zinc particulates in the syringe were gamma irradiated at a dosage of 25 kGy.

Immediately prior to use, zinc-HA gels of 1 mg/ml and 4 mg/ml were prepared with the following sequential dilution method. Zinc particulates were mixed with 2 ml of sterile HA by connection with a 3-way luer lock valve and using aseptic sterile techniques. Zinc particulates and HA were mixed 30 times to form a 10 mg/ml zinc particulate-HA gel. The 10 mg/ml zinc particulate-HA solution was further diluted to 1 mg/ml zinc particulate/HA by adding 0.2 ml of 10 mg/ml zinc particulate/HA to an additional 1.8 ml of sterile HA gel. A sterile 3-way valve was used to transfer both the 10 mg/ml zinc particulate-HA gel and HA gel into fresh 3 ml sterile syringes connected by a 3-way luer lock vlave. The resulting gel was mixed 30 times to provide a 1 mg/ml zinc particulate/HA gel.

Similarly, a 4 mg/ml zinc particulate-HA gel was obtained by adding 0.8 ml of the 10 mg/ml zinc particulate/HA to an additional 1.2 ml of sterile HA gel. A sterile 3-way valve was used to transfer both 10 mg/ml zinc particulate-HA gel and HA gel into fresh 3 ml sterile syringes connected by a 3-way luer lock vlave. The resulting gel was mixed 30 times to provide a 4 mg/ml zinc particulate/HA gel.

### Part B: Preparation of Zinc Powder-CMC Gel

Carboxyl-methyl-cellulose (CMC, 7HF PH) was obtained from Ashland Inc. (Wilmington, Del.). A 2.5% (w/v) aqueous solution of the CMC in phosphate buffer was prepared and sterilized via autoclaving. Zinc powder as described in Example 2, serving as the zinc particulates, was sterilized by gamma irradiation at a dosage of 25KGy. CMC gels containing 1 mg/ml and 0.25mg/ml zinc particulates, respectively, were prepared by mixing the sterile CMC gel and zinc powder using the sequential dilution method of Part A with the zinc-HA gel preparation, immediately prior to use.

### Example 6

CMC gels containing zinc particulates made in the manner of Example 5 were evaluated for reduction of tissue adhesion at a surgical site as follows.

Forty female New Zealand White rabbits, 2.4-2.7 kg, were used in the study. The rabbits were randomized into four treatment groups (table below) prior to initiation of surgery. Rabbits were anesthetized with a mixture of 55 mg/kg ketamine hydrochloride and 5 mg/kg Rompum intramuscularly. Following preparation for sterile surgery, a midline laparotomy was performed. The uterine horns were exteriorized and traumatized by abrasion of the serosal surface with gauze until punctate bleeding developed. Ischemia of both uterine horns was induced by removal of the collateral blood supply. The remaining blood supply to the uterine horns was the ascending branches of the utero-vaginal arterial supply of the myometrium.

At the end of surgery, no treatment, CMC vehicle control (4 ml), or CMC gel containing zinc particulates (4 ml) was administered. The horns were then returned to their normal anatomic position and the midline incision was sutured with 3-0 Vicryl suture. Table 6 summarizes the test conditions.

TABLE 6

| Group | | Animal |
|---|---|---|
| Number | Treatment | Number |
| Group 1 | Surgery Only | 10 |
| Group 2 | Vehicle Control (2.5% CMC gel) | 10 |
| Group 3 | 1 mg/ml Zinc Powder in 2.5% CMC gel | 10 |
| Group 4 | 0.25 mg/ml Zinc Powder in 2.5% CMC | 10 |
| | gel | |

After 21 days, the rabbits were terminated and the percentage of the area of the horns adherent to various organs determined In addition, the tenacity of the adhesions was scored. The results are shown in Table 7.

TABLE 7

| | Percentage | # Score |
|---|---|---|
| Group | Adhesion Free | ≦1.5/Total |
| Surgical Control | 0.0 | 0/10 |
| Vehicle Control | 21.25 | 3/10 |
| Zn Powder CMC | 32.5 | 8/10 |
| Gel (1 mg/ml) | | |
| Zn Powder Gel CMC | 26.25 | 4/10 |
| (0.25 mg/ml) | | |

No biocompatibility issues or adverse clinical observations were noted post-surgery. No inflammation was observed at necropsy. Finally, the zinc particulate loaded CMC gel formulation, surprisingly, showed reduction of adhesion at both non surgical and surgical sites, especially for the group tested with 1 mg/ml zinc powder CMC gel, which was significantly better than the control groups. It should be noted that CMC gel has an inherent anti-adhesion activity, and CMC is in a commercial anti-adhesion gel medical product.

### Example 7

CMC gels containing zinc particulates made in the manner of Example 5 were evaluated for reduction of tissue adhesion at a surgical site as follows.

Forty female New Zealand White rabbits, 2.4-2.7 kg, were used in the study. The rabbits were randomized into four treatment groups (table below) with n value of 10 for each group prior to initiation of surgery.

Rabbits were anesthetized with a mixture of 55 mg/kg ketamine hydrochloride and 5 mg/kg Rompum intramuscularly. Following preparation for sterile surgery, a midline laparotomy was performed. The uterine horns were exteriorized and traumatized by abrasion of the serosal surface with gauze until punctate bleeding developed. Ischemia of both uterine horns was induced by removal of the collateral blood supply. The remaining blood supply to the uterine horns was the ascending branches of the utero-vaginal arterial supply of the myometrium.

At the end of surgery, no treatment, CMC vehicle control (4 ml), or CMC gel containing zinc (4 ml) was administered. The horns were then returned to their normal anatomic position and the midline incision was sutured with 3-0 Vicryl suture. Table 8 summarizes the test conditions.

TABLE 8

| Group | | Animal |
|---|---|---|
| Number | Treatment | Number |
| Group 1 | Surgery Only | 10 |
| Group 2 | Vehicle Control (2.5% CMC gel) | 10 |
| Group 3 | 2 mg/ml Zinc Powder in 2.5% CMC | 10 |
| | gel | |
| Group 4 | 1 mg/ml Zinc Powder in 2.5% CMC | 10 |
| | gel | |

After 21 days, the rabbits were terminated and the percentage of the area of the horns adherent to various organs determined In addition, the tenacity of the adhesions was scored.

The results are shown in Table 9. Overall scores for the two treatment groups were different (p<0.01, ANOVA on ranks, Tukey's analysis for comparison between groups) from both surgical controls and vehicle controls. The zinc-containing CMC gels increased the number of adhesion free sites when compared with both the surgical and vehicle control (CMC gel). In addition, the number of animals with a score of 1.5 or less was increased in the zinc-containing CMC gel groups. Again, the test group which received 1 mg/ml zinc powder CMC gel had the best outcome.

These results demonstrate that particulates in the form of elemental zinc powder significantly reduced post-surgical tissue adhesion in an animal model (Rabbit DUH model). The zinc powder concentration of 1 mg/ml in the CMC gel appears to be close to the optimal dose for the application of post-surgical adhesion prevention under these testing conditions.

TABLE 9

| | Percentage | # Score |
|---|---|---|
| Group | Adhesion Free | ≦1.5/Total |
| Surgical Control | 1.25 | 0/10 |
| Vehicle Control | 11.25 | 0/10 |
| Zn Powder CMC Gel | 29.17 | 5/9 |
| (2 mg/ml) | | |
| Zn Powder CMC Gel | 43.75 | 9/10 |
| (1 mg/ml) | | |

### Example 8

Seventy-five male albino Wistar rats sold under the tradename SPRAGUE DAWLEY (CD [Crl:CD(SD)] strain), approximately 8 weeks old were randomized to 6 groups with 15 rats in each group. All animals in each group were anesthetized to effect with isoflurane and then administered an induction article once on Day 0 via intra-articular injection into the right ankle joint space. The induction article was a 50 microliter dose of Complete Freund's Adjuvant (CFA) with 2 mg/mL M. tuberculosis.

One treatment group served as the negative control and was untreated.

The animals of one treatment group were administered 50 microliters each of the 1 mg/mL zinc particulates in HA gel carrier prepared as described in Example 5 on day 5 through intra-articular injection.

The animals of another treatment group were administered 50 microliters each of the 4 mg/mL zinc particulates in HA gel carrier prepared as described in Example 5 on day 5 through intra-articular injection.

An additional group served as the vehicle control HA and received 50 microliters each of HA vehicle on the day 5.

Another treatment group was administered the positive control article morphine sulphate at a dose level of 3 mg/kg, once daily, prior to the functional measurements.

The effect of different treatments on pain relief was evaluated with an incapacitance test for weight bearing difference between the injected ankle and its counter lateral ankle as follows. Briefly, the weight borne on each hind paw was measured in triplicates employing a latency period of 5 s and the percentage weight borne on the affected right limb expressed. Measurements were made prior to the arthritis induction and at intervals beginning at Day 1, namely on Days −1, 1, 4, 7, 8, 10, 11, 13, 14, 19, 22, 25, and 28. The improvement in weight bearing percent on a given day of treatment was calculated by subtracting the baseline percentage of weight bearing (Day 4) . The mean improvement in deficiency was then obtained by averaging the improvement in weight bearing percent from all time points in each treatment group.

The mean improvement in weight bearing deficiency data demonstrate that single metal particulates at both 1 and 4 mg/ml in HA gel gave improvement in pain reduction in the affected paw, and the 4 mg/mL formulation showed improvement at a statistically significant level compared with the HA gel. The results are shown in Table 10.

TABLE 10

| | MEAN | |
|---|---|---|
| Deficiency Improvement | IMPROVEMENT | SE |
| Negative Control | 4.96 | 0.76 |
| HA Gel Vehicle Control | 5.03 | 1.08 |
| Zinc Powder-HA Gel 4 mg/ml | 9.24* | 1.07 |
| Zinc Powder-HA Gel 1 mg/ml | 7.44 | 1.39 |
| Morphine | 16.85 | 1.18 |

*Zinc-HA gel's efficacy is statistically significant vs. HA gel vehicle (F = 0.005, T - Test);
SE: Standard Error.

After CFA induction, all animals exhibited swelling in the right hind limb (CFA-induced paw) starting from Day 5 (data not shown). The rat joint swelling was determined by the measured volume difference between the CFA-induced paw and the control paw. Zinc particulates in the gel carrier showed a trend in improvement in the reduction of swelling when compared to negative HA carrier, and Morphine. The results are shown in Table 11. This result, consistent with the pain reduction efficacy observed suggests that zinc particulates in the HA gel carrier may be useful in pain relief in osteoarthritis.

TABLE 11

| | MEAN PAW | |
|---|---|---|
| | VOLUME | |
| | DIFFERENCE (ml) | SE |
| Negative Control | 0.72 | 0.11 |
| HA Gel Control | 1.24 | 0.23 |
| Zinc-HA Gel | 0.95 | 0.11 |
| 4 mg/ml | | |
| Zinc-HA Gel | 0.97 | 0.10 |
| 1 mg/ml | | |
| Morphine | 1.38 | 0.20 |

What is claimed is:

1. A therapeutic composition comprising single-metal particulates comprising a substantially pure, elemental metal having a Standard Electrode Potential of about −0.6V to about −2.5V and a carrier, said particulates having a particle size of about 10 nanometers to about 500 micrometers and being capable of generating a current density of less than 100 microA/cm².

2. The composition of claim 1, wherein said metal is selected from the group consisting of zinc, magnesium, and aluminum.

3. The composition of claim 1, wherein said particulates contain at least 90 weight percent of said metal.

4. The composition of claim 1, wherein said particulates comprise a mono-phase alloy comprising said metal and a secondary elemental metal.

5. The composition of claim 4, wherein said secondary elemental metal is selected from the group consisting of copper, iron, manganese, selenium and mixtures and compounds thereof

6. The composition of claim 1, wherein said carrier comprises an electrolyte.

7. The composition of claim 1, wherein said carrier is anhydrous.

8. A method of treating mammalian tissue, which comprises administering to said mammalian tissue a composition comprising single-metal particulates comprising a substantially pure, elemental metal having a Standard Electrode Potential of about −0.6V to about −2.5V and a carrier, said particulates having a particle size of about 10 nanometers to about 500 micrometers and being capable of generating a current density of less than 100 microA/cm².

9. The method of claim 8, wherein said administering is topical.

10. The method of claim 8, wherein said administering is oral.

11. The method of claim 8, wherein said administering is parenteral.

12. The method of claim 8, wherein said administering is intravaginal.

13. The method of claim 8, wherein said administering is intra-articular.

14. The method of claim 8, wherein said metal is selected from the group consisting of zinc, magnesium, and aluminum.

15. The method of claim 8, wherein said particulates contain at least 90 weight percent of said metal.

16. The method of claim 8, wherein said particulates comprise a mono-phase alloy comprising said metal and a secondary elemental metal.

17. The method of claim 16, wherein said secondary elemental metal is selected from the group consisting of copper, iron, manganese, selenium, and mixtures thereof

18. A method of treating a skin condition, which comprises topically applying to skin having such skin condition a composition comprising single-metal particulates comprising a substantially pure, elemental metal having a Standard Electrode Potential of about −0.6V to about −2.5V and a topical carrier, said particulates having a particle size of about 10 nanometers to about 500 micrometers and being capable of generating a current density of less than 100 microA/cm².

19. The method of claim 18, wherein said skin condition is acne or rosacea.

20. The method of claim 18, wherein said skin condition is a skin infection.

21. The method of claim 18, wherein said skin condition is skin aging.

22. The method of claim 18, wherein said composition further comprises an additional active agent.

23. The method of claim 22, wherein said additional active agent is selected from the group consisting of sunscreens, anti-wrinkling/anti-aging agents, antifungal agents, antibiotic agents, anti-acne, anti-psoriatic agents, and depigmentating agents.

24. A method of preventing tissue adhesion, which comprises applying to mammalian tissue that has been subjected to trauma or surgery a composition comprising single-metal particulates comprising a substantially pure, elemental metal having a Standard Electrode Potential of about −0.6V to about −2.5V and a carrier, said particulates having a particle size of about 10 nanometers to about 500 micrometers and being capable of generating a current density of less than 100 microA/cm².

25. The method of claim 24, wherein said administering is parenteral.

26. The method of claim 24, wherein said metal is selected from the group consisting of zinc, magnesium, and aluminum.

27. The method of claim 24, wherein said particulates contain at least 90 weight percent of said metal.

28. The method of claim 25, wherein said particulates comprise a mono-phase alloy comprising said metal and a secondary elemental metal.

29. The method of claim 28, wherein said secondary elemental metal is selected from the group consisting of copper, iron, manganese, selenium, and mixtures thereof

30. A method of reducing arthritis pain, which comprises applying to a joint suffering from arthritis a composition comprising single-metal particulates comprising a substantially pure, elemental metal having a Standard Electrode Potential of about −0.6V to about −2.5V and a carrier, said particulates having a particle size of about 10 nanometers to about 500 micrometers and being capable of generating a current density of less than 100 microA/cm².

31. The method of claim 30, wherein said administering is parenteral or intra-articular.

32. The method of claim 30, wherein said metal is selected from the group consisting of zinc, magnesium, and aluminum.

33. The method of claim 30, wherein said particulates contain at least 90 weight percent of said metal.

34. The method of claim 30, wherein said particulates comprise a mono-phase alloy comprising said metal and a secondary elemental metal.

35. The method of claim 34, wherein said secondary elemental metal is selected from the group consisting of copper, iron, manganese, selenium, and mixtures thereof

36. The method of claim 30, wherein said composition further comprises hyaluronic acid.

37. A method of reducing inflammation, which comprises applying to mammalian tissue suffering from inflammation a composition comprising single-metal particulates comprising a substantially pure, elemental metal having a Standard Electrode Potential of about −0.6V to about −2.5V and a carrier, said particulates having a particle size of about 10 nanometers to about 500 micrometers and being capable of generating a current density of less than 100 microA/cm².

38. The method of claim 37, wherein said administering is parenteral, topical, or oral.

39. The method of claim 37, wherein said metal is selected from the group consisting of zinc, magnesium, and aluminum.

40. The method of claim 37, wherein said particulates contain at least 90 weight percent of said metal.

41. The method of claim 38, wherein said particulates comprise a mono-phase alloy comprising said metal and a secondary elemental metal.

42. The method of claim 41, wherein said secondary elemental metal is selected from the group consisting of copper, iron, manganese, selenium, and mixtures thereof.

43. A method of treating microbial infection, which comprises applying to mammalian tissue suffering from microbial infection a composition comprising single-metal particulates comprising a substantially pure, elemental metal having a Standard Electrode Potential of about −0.6V to about −2.5V and a carrier, said particulates having a particle size of about 10 nanometers to about 500 micrometers and being capable of generating a current density of less than 100 microA/cm².

44. The method of claim 43, wherein said administering is parenteral, topical, or oral.

45. The method of claim 43, wherein said metal is selected from the group consisting of zinc, magnesium, and aluminum.

46. The method of claim 43, wherein said particulates contain at least 90 weight percent of said metal.

47. The method of claim 43, wherein said particulates comprise a mono-phase alloy comprising said metal and a secondary elemental metal.

48. The method of claim 47, wherein said secondary elemental metal is selected from the group consisting of copper, iron, manganese, selenium, and mixtures thereof.
